(19) United States
(12) Patent Application Publication
(10) Pub. No.: US 2007/0049514 A1
(43) Pub. Date: Mar. 1, 2007

(54) Resonance modulator for diagnosis and therapy

(76) Inventor: Lee Roy Roy Morgan, New Orleans, LA (US)

Correspondence Address: KLARQUIST SPARKMAN, LLP, 121 SW SALMON STREET, SUITE 1600, PORTLAND, OR, 97204, US

(21) Appl. No.: 10/547,223

(22) PCT Filed: Feb. 27, 2004
(86) PCT No.: PCT/US04/05845

§ 371 Date: Oct. 10, 2006

Related U.S. Application Data
(60) Provisional application No. 60/450,877, filed on Feb. 28, 2003.

(51) Int. Cl. A61K 38/16; A61K 31/7034; A61K 31/655; A61K 31/445; A61K 31/185; A61K 31/195; A61K 31/15

(52) U.S. Cl. 514/2; 514/317; 514/459; 514/432; 514/639; 514/114; 514/553; 514/561; 514/151; 514/23

(57) ABSTRACT

A method and device are disclosed for monitoring and/or altering an immune function. The method uses a resonance modulating compound as a coupling agent that is capable of interacting with the immune system to monitor or stimulate immune function. The resonance modulator has electromagnetic properties that attract immune cells to a target area to which the resonance modulating agent has been applied. Electromagnetic properties (such as a voltage amplitude) of the target region are altered in the presence of the resonance modulator, and serve as an indicator of immune function. An external stimulus (such as an applied electromagnetic field) can also be applied to the resonance modulator to enhance its immune stimulating and attractant properties. Particular examples of the resonance modulator are aryl hydrazones. The described aryl nitrohydrazones have the ability to interact with populations of cells with emergent behavioral characteristics associated with chemical, biological and radiation changes and injury. The disclosure also provides numerous other examples of resonance modulating agents, and provides attributes of these agents and assays for identifying additional resonance modulating agents.

### CROSS REFERENCE TO RELATED APPLICATION

This application claims the benefit of U.S. Provisional Patent Application No. 60/450,877 filed Feb. 28, 2003, which is incorporated herein by reference.

### FIELD OF THE INVENTION

This invention concerns methods of monitoring and altering immune function, for example in the diagnosis and/or treatment of infectious and neoplastic disease. It also discloses methods of treating infectious diseases, and tumors associated with infectious pathogens.

### BACKGROUND

1. Introduction

Immunotherapy involves the modulation of a subject's immune response to improve an innate ability to retain health. It has been recognized that there is a relationship between electromagnetic fields and biological functions, such as immunity. For example, U.S. Pat. No. 4,670,386 disclosed that the expression of strongly antigenic tumor specific antigens is induced by exposure of tumor cells to high frequency electromagnetic radiation that produces cyclic, rapid, alternating changes of polarity in dipolar molecular components of cancer cells. However, one of the drawbacks of very high frequency radiation is that it causes unwanted and potentially damaging heating of biological tissue.

The relationship between electromagnetic radiation and immunity is also illustrated by U.S. Pat. No. 6,038, 478, which discloses that lymphocytes can be attracted to a desired location in the body by applying electrodes to the desired location and stimulating the tissue with sufficient electric current to attract lymphocytes. Low energy alternating current magnetic fields were used to induce an immune response in U.S. Patent Publication 2002/0072646 A1. Similarly, WO 02/062418 A1 discloses enhancing immune function by exposing a subject to a magnetic field, or to very high frequency electromagnetic fields that are angularly modulated.

As noted in chapter 50 of The Biomedical Engineering Handbook (CRC Press 1995), biologic systems frequently have electric activity-associated with them. This activity can be a constant DC electric field, a constant flux of charge-carrying particles or current, or a time-varying electric field or current associated with some time-dependent or biochemical phenomenon. Bioelectric phenomena are associated with the distribution of ions or charged molecules in a biologic structure and the changes in this distribution resulting from specific processes.

Electromagnetic bioimpedence measurements have been used for diagnostic purposes. For example, WO 01/076475 discloses use of an alternating magnetic field to induce electrical eddy currents in biological tissue. An oscillator circuit is used to generate current in a coil adjacent targeted tissue. Since the amplitude of the resultant voltage is proportional to the conductivity of the tissue, changes in bioimpedence are used to detect changes in the tissue that are associated with tumors, such as prostate tumors. Another tissue impendence measuring device for differentiating tissue types is disclosed in WO 01/67098. The disclosure of both of these PCT publications (WO 01/076475 and WO 01/67098) is incorporated herein by reference.

The effect of electromagnetic fields on biological organisms and their cellular components has previously been appreciated. However, it has been difficult to take advantage of this inter-relationship, and there has been a need to more effectively couple the immune system to external sources of modulating electromagnetic radiation.

2. PTPs

The protein phosphatases are composed of at least two separate and distinct families: the protein serine/threonine phosphatases and the protein tyrosine phosphatases (PTs). Human protein tyrosine phosphatases (human PTPs) are a large and diverse family of proteins present in all eukaryotes. Each PTP is composed of at least one conserved domain characterized by an 11-residue sequence motif containing cysteine and arginine residues that are known to be essential for catalytic activities. The sequences of PTP share no similarity to serine or threonine, acid or alkaline phosphatases. The diversity in structure within the PTP family results primarily from the variety of non-catalytic sequences attached to the NH₂— or COOH— termini of the catalytic domain. There are numerous PTPs involved in intracellular phosphate metabolism and domains. The diversity of the extra cellular segments presumably reflects the variety of ligands to which the PTPs are exposed and catalyze phosphate transfer.

The protein tyrosine phosphatases (PTPs) are generally classified into two subgroups. The first subgroup is made up of the low molecular weight, intracellular enzymes that contain a single conserved catalytic phosphatase domain. All known intracellular type PTPs contain a single conserved catalytic phosphatase domain. Examples of the first group of PTPs include placental PTP 1B, T-cell PTP, rat brain PTP, neuronal phosphatase (STEP), and cytoplasmic phosphatases that contain a region of homology to cytoskeletal proteins.

The second subgroup of PTPs is made up of the high molecular weight, receptor-linked PTPs, termed R-PTPs, which include an intracellular catalytic region, a single tranembrane segment, and a putative ligand-binding extracellular domain. The structures and sizes of the putative ligand-binding extracellular “receptor” domains of R-PTPs are quite divergent, in contrast to the intracellular catalytic regions of R-PTPs which are highly homologous. All R-PTPs have two tandemly duplicated catalytic phosphatase homology domains, with the prominent exception of an R-PTP termed HPTPβ, which has only one catalytic phosphatase domain. (Tsai et al., J. Biol. Chem. 266(16):10534-10543 (1991)).

One example of R-PTPs is the leukocyte common antigen (LCA) (Ralph, S. J., EMBO J. 6:1251-1257 (1987)). LCA is a family of high molecular weight glycoproteins expressed on the surface of all leukocytes and their hemopoietic progenitors. A remarkable degree of similarity is detected with the sequence of LCA from several species (Charbonneau et al., Proc. Natl. Acad. Sci. USA 85:7182-7186 (1988)). LCA is referred to in the literature by different names, including T200, B220 for the B cell form, the mouse allotypic marker Ly-5, and more recently CD45 (Cobbold et al., Leucocyte Typing III, ed. A. J. McMichael et al., pp. 788-803 (1987)). CD45 is believed to play a critical role in T cell activation. These studies are reviewed in Weiss A., Ann. Rev. Genet. 25:487-510 (1991).

Another example of R-PTPs is the leukocyte common antigen related molecule (LAR) (Streuli et al., J. Exp. Med. 168:1523-1530 (1988)). In addition, published application W092/01050 discloses human R-PTP-α,β and γ and reports on the nature of the structural homologies found among the conserved domains of these three R-PTP and other members of this protein family.

The extracellular PTPs are related to surface recognition and adhesion molecules of leukocyte cell surface recognition. The PTPs are not only associated with human cells, but also present in prokaryotes and viruses, and bacteria. In the pathogenic bacterium Yersinia, the causative agent of bubonic plague, the Yop2b tyrosine-specific PTP is an essential virulence determinant.

Numerous studies have demonstrated the importance of PTPs in physiological processes. Phenotypic defects and hyperproliferative behavior of T-and B-lymphocytes, granulocytes and macrophages are considered to be key issues in the development of cancer and autoimmune diseases.

3. Resonance Structures

Resonance structures illustrate composite electronic structures of compounds in which the positions of electrons differ. Multiple alternative structures are said to be resonance structures, and a molecule is said to be a resonance hybrid of these structures.

### SUMMARY OF THE DISCLOSURE

A method is disclosed herein for stimulating a therapeutic response (such as an immune response) by administering to a subject in need of therapy (such as immunostimulation) a therapeutically effective amount of a resonance modulating compound that possesses resonating intramolecular dipole movements (or electrical densities) that allow it to interact with biological environments. Administration of the compound can take many forms, including topical application to a target area, insertion of pellets into the skin, placement in diseased organs, and inhalation. The resonance modulating compounds are capable of stimulating an immune response characterized in part by infiltration of immune cells, such as lymphocytes, into a target region in the vicinity of the resonance modulating compound. The electromagnetic properties of the target region also change as the immune cells enter the target area, and these electromagnetic changes can be detected (for example by electromagnetic signals provided by the resonance modulator) to measure the adequacy of a subject's immune response. Deficiencies of the immune response can be quickly detected in this manner, for example by the absence of an expected aggregation of immune cells, and appropriate therapeutic or preventative interventions taken. One such intervention is to expose the resonance modulating compound to an external electromagnetic stimulus that enhances the immune response both locally in the target area, and remotely throughout the body. Examples of such electromagnetic stimuli are electrical current flowing across an electrical potential through the compound, an induced magnetic field, or radiant energy (such as laser energy) applied to the compound.

Particular examples of the resonance modulator compound are aryl nitrohydrazones, such as phenylhydrazones, such as polyaryl mononitro- or dinitrophenylhydrazones, for example

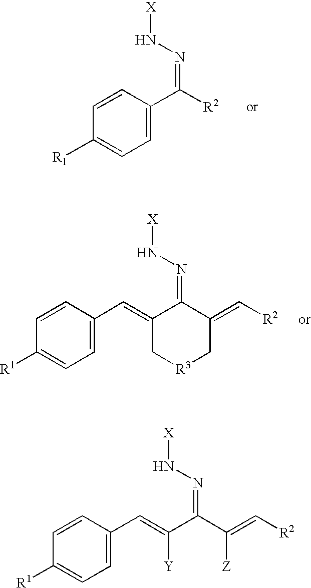

wherein R¹ is hydrogen, hydroxy, hydroxyphenyl (such as 2- or 4-hydroxyphenyl), acetate, phosphate, azido, nitrile, amino, dimethylamino, sulfate, methylsulfonate, phosphate, succinate;

R² is an unsubstituted (C₆H₅) or substituted phenyl group such C₆H₄OH, C₆H₄N₃, C₆H₄CN, 4-HO—C₆H₄—C₆H₄, C₆H₄OPO₂OH, C₆H₄OSO₂H, C₆H₄NH₂,C₆H₄NHMe₂, C₆H₄OSO₂Me, C₆H₄OCO(CH₂)ₓCO₂H, or C₆H₅Cl;

X is nitrophenyl such as C₆H₃-2,4(NO₂)₂, C₆H₄-4(NO₂), C₆H₄-3(NO₂), or C₆H₃-2,4(NO₂)₂;

R³═—O—, —S—, —CH₂—, —N—, —, —CHA- and —CHOA-; where A=aryl, ester, amide, lipid, carbohydrate, or peptide;

Y═H, (CH)ₓCH₃ (x=0-12), —S—CH₃, nitrile, amino, nitro, azido, succinate, or amide; and

Z=H, (CH)ₓCH₃ (x=0-12), —S—CH₃, nitrile, amino, nitro, azido, succinate, or amide.

In a particular example the compound is a 2,4-dinitrophenylhydrazone (referred to as A-007), wherein the compound is

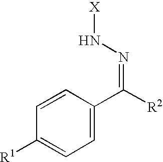

and R₁ is OH, R² is C₆H₄OH and X is C₆H₃-2,4(NO₂)₂. Many other examples of resonance modulating compounds are also disclosed herein, some of which are shown in the attached FIGS. 12, 16 and 17. These include formyl and acetylbarbituric phenylhydrazone analogs. Another example is 2,6-dibenzylidenecyclohexanone-2,4-trophenylhydrazone

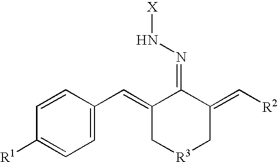

wherein X is a nitrophenyl (C₆H₃-2,4(NO₂)₂), R¹ is H, R² is unsubstituted phenyl, and R³ is CH₂.

These compounds possess resonating intramolecular dipole movements that are believed to be capable of electrostatic interaction with biological environments, and in particular examples interact with the extracellular catalytic receptors of R-PTP, such as those found on the R-PTP CD45+ PTP subtype, or with lymphocytes in general. This interaction attracts lymphocytes (such as T-lymphocytes) to target regions to which the compound has been applied. Although the resonance modulating compounds are very electronegative and capable of electrostatic affiliations, they typically lack substantial chemical reactivity and produce no local chemical reactions. They nonetheless appear to activate immune function, for example by mobilizing lymphocytes (for example T lymphocytes such as CD45+ T-lymphocytes, for example CD45RO+ or CD45RA+ lymphocytes) from lymphatic networks, and concentrate immunological activity in the targeted regions of the body that have been exposed to the compound. The resonance modulator compounds are also capable of stimulating the immune system at locations remote from the targeted region, for example in the spleen, by its interactions with the distributed networks of immune cells, such as those found in the lymphatic system. This mobilization of immune response can be used to treat infections or tumors.

In particular embodiments, the resonance modulator compound is administered by applying it to the skin of the subject, for example by applying it topically in a gel to the surface of the skin, or otherwise introducing it into the skin, for example by intradermal placement of crystals or pellets of the compound. However the resonance modulator can also be administered by introducing the compound into the body, for example into a diseased organ or a tumor (such as a malignant tumor or metastatic lesion), to stimulate a local immune response, mobilize lymphocytes from the lymphatic system, and direct an immune response at the target organ or tumor. The resonance modulator can also be administered in an aerosol preparation for tracheobronchial or oropharyngeal administration. In one example, the compound is applied topically to or adjacent a metastatic or epithelial lesion, such as a chest wall breast cancer lesion or a cervical epithelial carcinoma. The compound is applied to the epithelial or epidermal surface of the subject on an area that is in therapeutically sufficient proximity to the lesion to stimulate the immune response in the target region. In the example of a superficial lesion (such as a chest wall recurrence of breast tumor or a cervical or anal epithelial cancer), the compound is applied directly to the lesion.

The resonance modulating activity (and in turn an immune stimulating activity or PTP activating activity) of the compound can be enhanced by exposing the compound to an electromagnetic field (such as a time varying electric field, a time varying magnetic field, and/or a radiating electromagnetic field) that induces increased resonance modulation of the compound. For example, such a field can be induced by placing a magnetic probe in the vicinity of the compound that has been administered to the subject, or by providing a current that flows through the compound between two electrodes, or by a laser that irradiates the compound with laser energy. In particular embodiments, activity of the immune system can be conveniently up-regulated by applying the resonance modulator compound to a target region of the skin using a convenient patch or pellet, and inducing the electromagnetic field in a manner that increases the resonance modulation of the compound. In this manner, the resonance modulator on the skin surface acts as a convenient coupler between the externally applied electromagnetic field and the immune system, which can increase immune function particularly locally at the site of the resonance modulator. However, it is also believed that immune function can be enhanced systemically, remote from the resonance modulating compound.

The ability of the resonance modulator to interact with PTPs (such as components of the immune system) also allows the compound to be used to monitor immune function. It has been observed that application of the resonance modulator to a target region (for example in a patch applied to the skin) mobilizes PTP expressing cells, such as cellular components of the immune system-L The attraction of the immune cells apparently occurs by electrostatically interacting with and attracting dendritic cells and other early phase lymphoid cells. These cells aggregate in the vicinity of the resonance modulator, and change electromagnetic characteristics (such as bioimpedence) in the target region in which they aggregate. The changed electromagnetic characteristics are detectable, for example, as a change in amplitude of the voltage difference detectable across the target area, even in the absence of an applied voltage potential across the electrodes. In particular embodiments, electrodes are placed in contact with the modulator, and the inherent varying voltages are produced by resonance modulation of the compound over time. The amplitude of these varying voltage potentials is used to monitor immune function. For example, a reduction in voltage amplitude between the electrodes as compared to a normal control is an indication of impaired immune function.

The amplitude of the waveform (or other electromagnetic characteristics) may be monitored to determine a response of the subject's immune system to the compound. An increase in the amplitude of the voltage over time indicates that an immune response has occurred (and that immune effector cells such as lymphocytes have migrated to the target region). If the increase in amplitude falls below a predetermined threshold (such as an expected increase of at least 10%, 25% or 50%), then further diagnostic or therapeutic interventions may be undertaken to assess or correct the reasons for the impaired immunity. For example, a rigorous search can be undertaken for infectious, toxic or neoplastic causes of the impaired immune response. Alternatively, the immune response can be upregulated by exposing the resonance modulating compound to an electromagnetic field that induces increased resonance modulation of the compound. Another approach would be to initiate treatment of the subject with appropriate anti-infective or anti-neoplastic chemotherapeutic agents.

Resonance stimulating agents are particularly effective for treating a tumor, by administering the resonance stimulating agent to the subject (for example by topical application to a target region over or adjacent a cutaneous metastasis). An external electromagnetic field may then optionally be applied to the agent to increase its resonance modulation and thereby increase its immunostimulant effect. The external electromagnetic field may be produced, for example, by a magnetic probe that induces a localized magnetic field, an induced external current applied across electrodes, or a laser that stimulates resonance modulation of the resonance modulating compound. Resulting increased mobilization of antigen presenting cells (such as dendritic cells) and immune effector cells (such as T-cells) to the target region helps direct immunosurveillance and immune effector activity to the tumor target. Resonance modulating agents can be used, for example, to treat a cervical, anal or vaginal carcinoma, such as a carcinoma associated with an HPV infection in which HPV has induced a malignant transformation of the cells.

In particular embodiments, the immune response is stimulated either through or to a RPTP expressing cell, such as a CD45+ cell, such as a CD45+ T-lymphocyte or a RPTP or CD45+ expressing infected cell. An example of such a cell would be a lymphocyte produced in response to a virally infected cell, such as a cell infected with papillomavirus, for example human papillomavirus (HPV), or an immunodeficiency virus (such as HIV).

In particular methods of monitoring an immune response, the resonance modulating compound is placed in contact with the subject (for example on or in the skin of the subject) so that the inherent resonance modulation of the compound produces characteristic waveforms of the compound. The waveforms (such as alternation of voltage potentials or other electromagnetic waves) detected from the compound are monitored to detect waveforms produced by the compound that are associated with altered immune function. In particular embodiments, the waveforms are monitored by detecting voltage changes in the compound over time, for example by detecting waveforms that have a decreased amplitude from an expected increase that would be seem over time. In some embodiments of the method, once the waveforms have been detected that are associated with altered immune function, a diagnostic or therapeutic intervention may be undertaken in response to the detection of the waveforms.

For example, the intervention can be a diagnostic intervention designed to detect a specific feature of the altered immunity (such as decreased number or function or particular cells involved in the immune response, such as T-lymphocytes) or a cause of the altered immunity (such as infection with an immunodeficiency virus, or the presence of a neoplastic condition, such as a tumor, that alters immune function). Alternatively, detection of altered immunity results in a therapeutic intervention, such as administration of an anti-neoplastic or anti-infectious therapy (such as an antibiotic or antiviral drug), and/or modulating resonance of the compound to enhance immune function. Modulating resonance of the compound can include applying an induced electromagnetic field to the compound that enhances resonance modulation of the compound, which in turn improves immune function. In particular embodiments, the improved immune function constitutes improved mobilization of T-lymphocytes (such as CD45+ T-lymphocytes) to the site of application of the resonance modulating compound.

The disclosed methods also include a method of treating a tumor by exposing the tumor to a therapeutically effective dose of a resonance modulating compound, and applying an external electromagnetic field to the compound to increase its resonance modulation of the compound and thereby increase an anti-tumor effect of the compound to treat the tumor. Alternatively, the method can be used to treat an infection (such as a bacterial infection like Yersinia pestis infection or a viral infection, such as an HPV or HIV infection, or other infections in which a PTP is expressed by the pathogen) by administering a therapeutically effective amount of the compound In particular examples, the therapeutic amount is sufficient to interact with a PTP extracellular receptor of a cell to activate the receptor. In particular examples of either treatment, the compound is applied to the skin of the subject, or to an area of infection or neoplasia. The compound may, for example, be applied as a topical gel to the skin of the subject, or to urogenital or anogenital epithelium (such as anal, vaginal or cervical epithelium) that is infected with a papillomavirus (such as HPV). In some examples, the epithelium is dysplastic or metaplastic epithelium, such as cervical intraepithelial or high grade or squamous intraepithelial neoplasms (CIN/HSIL), anal intraepithelial neoplasms (AIN/HSIL) or a squamous carcinoma. In particular examples, the compound is applied as a 0.25% gel that is applied topically to the subject for a period sufficient to have a therapeutic effect, such as at least five days. In particular methods of treatment, gel is applied daily, and/or the effective amount is at least 2 grams of a topical gel containing at least 0.25% of the compound.

The unusual properties of the resonance modulating agent also permit it to be used in methods of concentrating dendritic cells and/or lymphocytes, either in vitro or in vivo. For example, RPTP+ cells, such as lymphocytes (for example CD45+ cells, such as T-lymphocytes) can be concentrated from biological tissue (or in culture) by exposing the biological tissue (or cells) to an effective amount of the resonance modulating agent In one example, that agent is applied to the skin of a subject to concentrate RPTP+ cells (such as lymphocytes) at and around the site of application of the agent. However, RPTP+ cells, such as lymphocytes and/or dendritic cells, can be attracted to any target region of the body in which the agent is introduced, such as a breast, colon or prostate tumor. Alternatively, the agent can be introduced into a tissue culture that contains RPTP+ cells such as lymphocytes and/or dendritic cells to attract them to the agent Selective concentrations of sub-populations of cells can be achieved in this manner. The resonance modulating agent can also be used to increase the concentration of CD45RO+ and CD45RB+ cells, for example by inducing expression of these cell surface markers. In particular embodiments, selective expression of CD45RO+ occurs, which is involved in cytotoxic activity.

The foregoing and other objects, features, and advantages of the invention will become more apparent from the following detailed description of several embodiments which proceeds with reference to the accompanying figures.

### BRIEF DESCRIPTION OF THE FIGURES

FIG. 1 is a schematic illustration of the interaction between a 4,4′-dihydroxybenzophenone-2,4-dinitrophenylhydrazone (A-007) and the CD45+ T-lymphocyte surface receptor.

FIG. 2 illustrates the structural formula of A-007.

FIG. 3 is a schematic illustration of the resonance structures of A-007.

FIG. 4 is a digital image illustrating the linear alignment of A-007 crystals that have been crystallized in a magnetic field.

FIG. 5 is a digital image illustrating a tissue culture of lymphocytes (top) and the same culture of lymphocytes after one hour in the presence of a crystal of A-007.

FIG. 6 is a screen print from an oscilloscope showing changes in voltage amplitude of lymphocytes in tissue culture before exposure to A-007 (top) and after one hour of exposure to A-007. The amplitude of the voltage increased from about 1.4 volts to about 1.8 volts following one hour of exposure to A-007 in culture.

FIG. 7 is a schematic diagram illustrating the relationship of the lymphatic system to the skin, and further illustrating an oscilloscope device for monitoring bioimpedance and inducing electromagnetic fields that interact with a resonance inducing agent applied to the skin of a subject.

FIG. 8 is a digital image of a patch of A-007 in a gel for application to the skin. The patch is provided with electrically conductive leads to which electrodes are connected, for monitoring electromagnetic properties of the gel (such as patch impedance) after it is applied to the skin. The electrodes are also available for connection to positive and negative electrical leads to introduce a current through the patch for the purpose of tuning the resonance modulator.

FIG. 9 is a screen print from an oscilloscope, illustrating changes in a voltage waveform over time through the patch of FIG. 8 applied to a normal subject. The patch contains 0.5 g of A-007 as a 0.25% gel applied to a 2×2 cm skin area on the back of a subject. The top waveform is taken immediately after the A-007 patch is applied to the skin. The bottom waveform is taken after the patch has been continuously in place for seven days. During this time, the amplitude of the voltage waveform has increased by more than 25%, from about 3 volts to about 4 volts. This is an example of an expected increase in amplitude that would be expected in a normal person, or in this particular person as a baseline value.

FIG. 10 is a digital image of a cervical biopsy, showing the response of cervical cancinoma-in-situ and CIN to topical application of A-007 to the cervix. The image on the left shows nests of malignant cells prior to application of topical A-007. The image on the right shows the radical organization of the epithelium with loss of malignant changes after 5 days of treatment with topical A-007.

FIG. 11 is a screen print of energy generated from A-007 crystals over time, which illustrates intermittent changes in frequency output (frequency excursions) that are believed to be associated with different resonance states.

FIG. 12 is a schematic drawing that shows the chemical structures of a variety of resonance modulator compounds (referred to as compounds 1-37), as well as the synthesis scheme for compounds 12-37.

FIG. 13 is a digital image that shows hyperplastic HPV infected cervical epithelium (cervical intraepithelial neoplasia or CGN) both before and after treatment The infected epithelium was treated topically with 2 grams of a 0.25% A-007 gel per day for 5 days. The upper left (pre-treatment) photograph shows staining for CD45RO+ lymphocytes (activated), which is very faint; whereas the photograph on the upper right shows that, after five days of topical A-007 intravaginal treatments, the CD45RO+ lymphocyte (activated) population becomes very intense. The bottom photographs compare the CD45 RA+/RO+ ratio after five days of topical A-007 (same dosing as above) showing that the RO+ cell component (the active variant which implements cytotoxic activity) is the predominant form. The RO+ variant is required for cytotoxic activity.

FIG. 14 is a digital image that shows HeLa cancer cells incubated with crystals of A-007 for 24 hours. The clear cells to the right of the crystal are attracted toward the crystal in the middle of the photograph. Upon contact with the crystal the cells migrate away, undergo pyknosis (DNA agglutination and necrosis) and die. This figure is believed to demonstrate the attraction of the cell surface membranes to the resonating intramolecular magnetic patterns of the A-007 crystals, and provides clear evidence of the antitumor effect of the crystals.

FIG. 15 is a digital image that shows the effects of BDP-DNP on cell growth for a malignant squamous cell cancer (SCCA-HM) growing in tissue culture (pre-post Rx). On the left are large lacey star shaped squamous cancer cells associated with small white clumps of naïve T-lymphocytes. After 24 hours incubation with BDP-DNP (0.4 mcg/mL) in tissue culture media (as in U.S. Pat. No. 5,270,172), the lymphocytes underwent an impressive stimulation (large white clumps of activated T-lymphocytes) and destruction of the cancer cells. The dark background seen on the right is due to the color of the drug. All the fluffy white cells are large clumps of activated lymphocytes. No cancer cells are detected in the post-treatment photograph.

FIG. 16 is a schematic drawing of 2,6-dibenzylidenecyclohexanone-2,4-dinitrophenylhydrazone (structure 38) which has excellent binding affinities to PTPCD 45 receptor, and upregulates the receptor.

FIG. 17 illustrates the interaction of 2,6-dibenzylidenecyclohexanone-2,4-dinitrophenylhydrazone (structure 38) with PTP CD45+ receptor.

FIG. 18 illustrates the binding of A-007 (2,4-DNP) and other ligands to the CD45 receptor on T-lymphocytes, which results in receptor dimerization and subsequent signaling. Dimerization initiates a variety of effects, including maturation of the cell and upgrading of function.

### DETAILED DESCRIPTION

### I. Abbreviations

A-007: 4,4′-dihydroxybenzophenone-2,4-dinitrophenylhydrazone

AMTR: A-007 Magnetic Transistor Resonator

An: Antigen

APC: Antigen Presenting Cell

BDP-DNP: 2,6-Dibenzylidenecyclohexanone-2,4-dinitrophenylhydrazone

DC: Dendritic Cell

DNP: Dinitrophenylhydrazone

PTP: Protein Tyrosine Phosphatase

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

If any of these terms conflict with a document that has been incorporated by reference, the meanings of terms set forth in this document will control.

In order to facilitate review of the various embodiments of this disclosure, the following explanations of specific terms are provided.

Animal: Living multi-cellular vertebrate organisms, a category that includes, for example, mammals and birds. The term mammal includes both human and non-human mammals. Similarly, the term “subject” includes both human and veterinary subjects.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes.

CD Markers: Cluster of differentiation (CD) markers that can serve as cell surface markers for different classes of lymphocytes. A systematic nomenclature has been developed in which CD has been determined by the binding of certain monoclonal antibodies to certain human leukocyte antigens (HLA). Further information about CD markers is disclosed, for example in Roitt et al., Immunology (6^{th} Edition), 2001. A certain subset of CD marker is the CD45 marker, which is a leukocyte common antigen (LCA). CD45 RA, CD45RB and CD45RO are restricted LCAs that are a subset of CD45+ cells, and are further described in Roitt, et al., Appendix 2. Certain characteristics of these LCAs are shown in the following table:

| Name | Expression | M.W. | Functions | Other names |
|---|---|---|---|---|
| CD45 | leukocytes | 180-240 | tyrosine phosphatase, | Leukocyte |
| | | | augments signaling | common antigen |
| | | | through antigen | (LCA), T200, B220 |
| | | | receptor of B and T | |
| | | | cells, multiple isoforms | |
| | | | result from alternative | |
| | | | splicing (see below) | |
| CD45RO | T cell subsets, B | 180 | isoform of CD45 | |
| | cell subsets, | | containing none of | |
| | monocytes, | | the A, B and C exons | |
| | macrophages | | | |
| CD45RA | B cells, T cell | 205-220 | isoforms of CD45 | |
| | subsets (naive T | | containing the A exon | |
| | cells) monocytes | | | |
| CD45RB | T cell subsets, B | 190-220 | isoforms of CD45 | T200 |
| | cells, | | containing the B exon | |
| | monocytes, | | | |
| | macrophages, | | | |
| | granulocytes | | | |

Chemotaxis: Movement of an organism or a single cell, such as a leukocyte, in response to a chemical compound. As used herein, chemotaxis can occur in response to any physical property of the compound, including electrostatic properties.

Chemotherapy; chemotherapeutic agents: As used herein, any chemical agent with therapeutic usefulness in the treatment of diseases, for example diseases characterized by abnormal cell growth, such as neoplasms. In one embodiment, a chemotherapeutic agent is an agent of use in treating neoplasms such as solid tumors. One of sill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St Louis, Mosby-Year Book, 1995; Fischer D. S., Knobf M. F., Durivage H. J. (eds): The Cancer Chemotherapy Handbook, 4th ed. St Louis, Mosby-Year Book, 1993).

Dendritic cell (DC): Dendritic cells are the principle antigen presenting cells (APCs) involved in primary immune responses. Dendritic cells include plasmacytoid dendritic cells and myeloid dendritic cells. Their major function is to identify and process antigen in tissues, migrate to lymphoid organs and present antigenic information in order to activate the T-cell cascade. Immature dendritic cells originate in the bone marrow and reside in the periphery as immature cells.

DCs are capable of evolving from immature, antigen-capturing cells to mature, antigen-presenting, T cell-priming cells; converting antigens into immunogens and expressing molecules such as cytokines, chemokines, costimulatory molecules and proteases to initiate an immune response.

Hydrazone: A compound with the structure R₂C═NNR₂, differing from a ketone or aldehyde by the replacement of the double bonded oxygen with the ═NNR₂. A hydrazone is generally formed by the condensation of a hydrazine with a carbonyl group. An aryl hydrazone is a hydrazone in which at least one of the R groups is an aryl group, for example a phenyl group (a phenylhydrazone). A nitrophenylhydrazone is a phenylhydrazone having one or more NO₂ substitutions on the phenyl ring.

Immune response: A response of an organism to a foreign (non-self) agent. An immune response to a stimulus is implemented by cells of the immune system, such as a B-lymphocyte, or a T-lymphocyte. In one embodiment, the response is specific for a particular antigen (an “antigen-specific response”).

Infectious agent: An agent that can infect a subject, including, but not limited to, viruses, bacteria, and fungi.

Inherent electromagnetic waveforms: Waveforms that are produced as a characteristic of a compound, independent of actively induced electromagnetic phenomena, such as intentional application of electrical currents, electrical potentials, or magnetic fields. An example of a waveform is the waveform produced by alternating voltages over time, for example waveforms that alternate between a positive and negative potential, often in a predictable manner (for example as defined by a sine wave). Waveforms can be monitored by a variety of electromagnetic monitoring devices, such as a volt meters that measures an electrical potential across two electrodes in contact with the compound.

Intervention: An intervention is an action taken to detect or affect a physiologic or medical state of a subject A diagnostic intervention detects the state of the subject, for example by performing a laboratory test, such as a blood test, biopsy, imaging study or physical examination. A therapeutic intervention affects the state of the subject, for example by performing surgery, administering a drug or other treatment, or performing any other therapeutic procedure.

Isolated: An “isolated” biological component (such as a dendritic cell or lymphocyte, or a population of those cells) has been substantially separated or purified away from other biological components in the cell of the organism in which the component naturally occurs.

Laboratory evidence of impaired immunity: Objective laboratory data that is generally medically accepted as evidence of reduced immunity, such as a white blood cell count that is below accepted norms in a particular laboratory, reduced lymphocyte (such as T-lymphocyte) concentration, or evidence of generalized impaired activity of any cell of the immune system. In particular examples, the data demonstrate impaired cellular immunity or humoral immunity, or both

Lacking chemical reactivity: Certain resonance modulating compounds are electrostatically active but substantially non-reactive under physiological conditions (in the body) because of their resonance stabilization. Such lack of chemical reactivity results in the compound being excreted substantially completely unchanged after systemic (such as oral or intravenous) administration. An example of this lack of reactivity is seen, for example, by an absence of N-methylation of A-007 in the body

Leukocyte: Cells in the blood, also termed “white cells,” that are involved in defending the body against infective organisms and foreign substances. Leukocytes are produced in the bone marrow. There are 5 main types of white blood cell, subdivided between 2 main groups: polymorphonuclear leukocytes (neutrophils, eosinophils, basophils) and mononuclear leukocytes (monocytes and lymphocytes). When an infection is present, the production of leukocytes increases.

Neoplasm: An abnormal cellular proliferation, which includes benign and malignant tumors, as well as other proliferative disorders.

Papillomavirus: Papillomaviruses are small, nonenveloped viruses with an icosahedral symmetry, capsomere, and a double-strand circular DNA genome of about 8,000 bp. All papillomaviruses have a similar genetic organization. The viral genome is divided into an early region which encodes the genes required for viral DNA replication and cellular transformation, a late region that codes for the capsid proteins, and a regulatory region that contains the origin of replication and many of the control elements for transcription and replication.

Papillomarviruses have a high degree of species specificity. There are no known examples of natural transmission of human papillomavirus (HPV) to other species. Papillomaviruses also display a marked degree of cellular tropism, infecting only surface squamous epithelia of the skin or mucosa and producing for the most part benign epithelial tumors. Specific viral types appear to have a preference for either cutaneous or mucosal types. For example, HPV-11 does not readily infect cutaneous epithelium from other body sites but can infect mucosal epithelium of either the genital or the respiratory tract. However the papillomaviruses induce cellular proliferation and transformation that can lead to the development of invasive cancers. HPV infections have been associated with the development of cervical and anal cancers.

Pharmaceutical agent or drug: A chemical compound or composition capable of inducing a desired therapeutic or prophylactic effect when properly administered to a subject. Pharmaceutical agents include, but are not limited to, chemotherapeutic agents and anti-infective agents.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of manoitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Purified: The term “purified” does not require absolute purity; rather, it is intended as a relative term. Thus, for example, a purified preparation of lymphocytes is one in which the lymphocytes are present in a greater concentration than in its natural environment within the body.

Resonance Modulator: A compound that possesses resonating intramolecular dipole movements (or electrical densities) that allow it to electrostatically interact with biological environments. A resonance modulator is also characterized by the emission of oscillating frequency waves generated by the compound's intramolecular resonance. This resonance is believed to convey the electrical dispositions for interactions with cells of the immune system (such as dendritic cells) to up-regulate and/or enhance immunity. Resonance modulators are capable of attracting immune cells, concentrating them in a target region of the peripheral immune system adjacent the resonance modulator, and in some instances have a distant effect on circulating immune cells (such as immune cells in the peripheral blood and lymphoid tissue such as a lymph node or the spleen). Many resonance modulators have a crystalline structure. Examples of assays for selecting resonance modulator candidates are disclosed in Example 14.

Sigma waves: Sigma waves that are recorded from crystals and tissue culture are waves over and above the base line of electrical energy that is generated from the heart and other biorhythms within the body. The sigma waves therefore measure voltage changes with time and amplitude increases with time after exposure to A-007.

Subject in need of immunostimulation: A subject having a condition that would benefit for general or specific stimulation of the immune system. Examples include subjects with immune deficiencies (such as persons infected with HIV or who have recently received chemotherapy or other immunosuppressive drugs), and persons with conditions that could be improved by the stimulation of an immune response (such as subject infected with a pathogen or tumor that alters immune function, such as a lymphoma). In certain examples, the subject is in need of immunostimulation for a condition other than a tumor, for example because of infection or immunocompromise (for example infectious or pharmaceutically induced inmnunodeficiency).

Therapeutically effective dose: A dose sufficient to inhibit or prevent advancement, or to cause regression of the disease, or which is capable of relieving symptoms caused by the disease, such as pain or swelling.

Waveforms associated with altered immune function: Waveforms that are noted to be present in subject having an enhanced or decreased immune function. For example, the amplitude of voltage potential waveforms increases over time after a resonance modulating compound is applied to a subject. The increase in amplitude is a consequence of normal immune function, and is indicative of the mobilization of immune cells (such as lymphocytes) and their migration to the site of application of the compound. However, an increase in amplitude that is below that seen in the same subject at a baseline measurement taken during health, or an increase below a range that is statistically normal in a population of subjects, can be taken as associated with altered (impaired) immune function. Conversely, an increase in amplitude that is greater than normal (as determined for a particular individual or a population) is an indication of supra-normal immune function.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” In addition, the materials, methods, and examples are illustrative only and not intended to be limiting.

### III. Descriptions of Several Embodiments

The methods disclosed herein concern the use of resonance modulators to act as an activator of PTPs. In certain examples, the resonance modulator acts as an imnmunostimulant and/or as a coupling agent between the immune system and external monitors or modulators. The resonance modulators are believed to interact with PTPs, for example interacting with cellular components of the immune system (such as CD45+ receptor lymphocytes or dendritic cells) to promote maturation of immune cells, and recruitment of other cellular components of immunity in an immune response. The ability of resonance modulators to attract immune cells to the vicinity of the modulator also allows an immune response to be directed to a target region within the body (such as a tumor) where an immune response is needed for treatment of a localized condition. However, the resonance modulators are also capable of eliciting an immune response at distant sites, such as remote lynmphatic tissue or metastatic lesions. The resonance modulators therefore provide a novel interface with the immune system that allows information about immune status to be collected and interventions (including manipulation of immune function) to be performed.

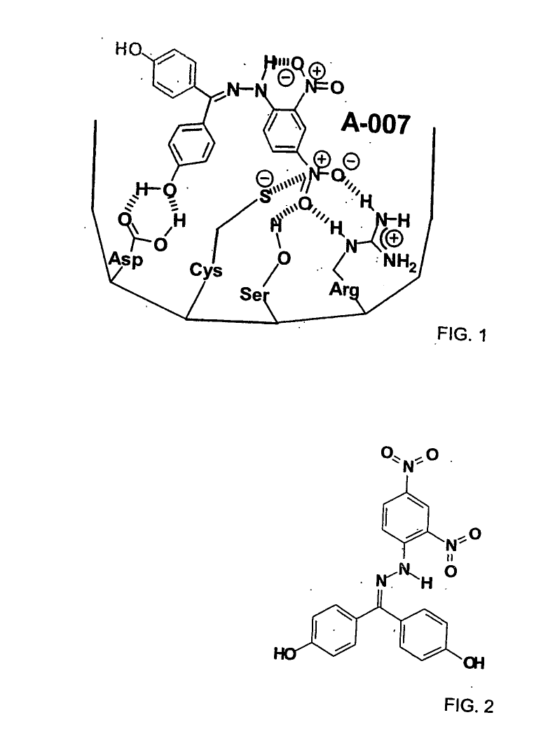

An example of a resonance modulator is 4,4′-dihydroxybenzophenone-2,4-dinitrophenylhydrazone, also known as A-007, the structure of which is illustrated in FIG. 2. This compound is a particularly suitable substance to act as a sensor and immune modulator, because it lacks chemical reactivity, participates in no local chemical reactions, and has both an electronegative ground state and an affinity for cell membrane receptors. An example of its affinity for RPTP+ cell membrane receptors is the interaction between A-007 and the CD45+ T-lymphocyte surface receptor, which is illustrated in FIG. 1. These interactions are believed to induce maturation of the cells with which they interact, to promote the immune response.

### EXAMPLE 1

### Resonance Modulation with Phenylydrazones

A variety of compounds are disclosed in this Example that are capable of acting as immune modulators. A particular suitable substance(s) having both modulating and sensor properties is a polyaryl mononitro- or dinitrophenylhydrazone such as 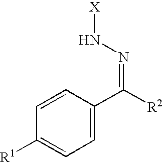
wherein R¹ is hydrogen, hydroxy, 2- or 4-hydroxyphenyl, acetate, phosphate, azido, nitrile, amino, dimethylamino, sulfate, methylsulfonate, phosphate, nitroso, succinate or another water soluble electrophilic group capable of hydrogen bonding; R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; and X is C₆H₃-2,4(NO₂)₂, C₆H₄-4(NO₂), C₆H₄-3(NO₂), or C₆H₃-2,4(NO₂)₂.

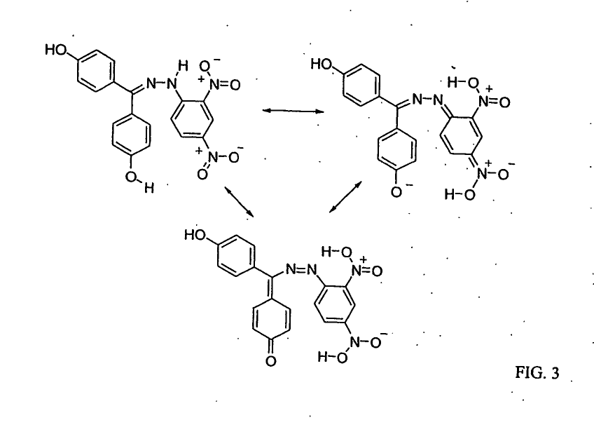

As illustrated in FIG. 3, these chemical structures are able to resonante through multiple dipolar configurations of conjugated electron rich/poor areas that allows/permits migrating intermolecular hydrogen bonding, as well as nucleophilic/electrophilic attractions with complementary sites to exist for moments in time. As illustrated in FIG. 1, these resonating mini-dipoles are capable of attracting for example —SH, —COOH, —NH₂—CH—NH₃⁺ that are present in amino acids (such as cysteine, threonine, arginine), carbohydrates (muccopolysaccharides) and glycoproteins, all of which are essential components of cellular regulators in the immune system For example, simple interactions with 3 or 4 amino acids on the surface of the CD45+RA receptor on a lymphocyte is believed to induce the expression of a modified surface protein (RO, RB) capable of initiating the T-ell cascade with influxes of CD4+/CD8+ into tissues, organs and the circulation. In the “moment in time” during which the hydrogen bonding and attractions are modulated via electrical stimulation, the transient electrostatic interactions that intrinsically exist with these structures are increased and can be quantitated with NMR spectroscopy.

The presence of multiple electronegative moieties capable of multiple resonance structures, attached through conjugated and aryl bonds, generates oscillating frequency waves, such as those shown in FIG. 11. Certain resonance modulators described herein (such as A-007) have a sufficient “electron count” in that they have an excess of electrons for electrostatic interactions. The excess allows attraction of electrophilic/nucleophilic centers that are present in proteins and other biololecules, and they do not need to react (via a transfer of electrons and covalent bond formations) for structure stabilization. Instead, simple “flirtations” with the environment are believed to induce changes in other polymolecular configurations, such as the CD45+ receptor, without changing the structure of the resonance modulator.

In a particularly disclosed embodiment of the resonance modulator, R¹ is OH, R² is C₆H₄OH and X is C₆H₃-2,4(NO₂)₂, which is 4,4′-dihydroxybenzophenone-2,4-dinitrophenylhydrazone (A-007). This compound is highly electronegative, and exists in several resonance forms, as illustrated in FIG. 3. Representative oscillating frequency waves generated by the hydrazones electrostatically interact with dendritic cells and other early phase lymphoid cellular elements to promote immunity. The interactions can be incorporated into one or more Dirac equation(s) to formulate quantum molecular changes in the skin associated with influxes of potentially selective and diagnostic lymphocyte components.

Stabilization of certain resonance forms of A-007 is believed to occur, which results in alteration of frequency and electrical effluxes from the test areas as the resonance modulator undergoes electrostatic interactions with lymphocytes. The cycle of resonance will be disturbed by this interaction, and such interference has been monitored (for example by detecting changes in waveforms of voltage over time) and used as an indicator of activity. Variations in the waveform patterns indicate the presence of an evolving cellular immune response (such as the infiltration of dendritic cells and lymphocytes). An absence of expected waveforms or other detected patterns of interference are also useful for detecting an abnormal immune response that requires further investigation and/or treatment. Hence the resonance modulator acts as a coupling agent between immune activity and an external monitor.

The resonance modulator is capable not only of detecting cellular immune activity, but it also couples the immune system to external physical modulators that can be used to alter this activity. For example, a magnetic resonator probe may be used to apply a magnetic field to the surface of the skin, for example by applying the probe to or adjacent the skin surface. The magnetic probe activates the A-007 crystals, stimulates resonant energy efflux and promotes tissue T-cell contact. Thus, if an individual is not able to induce an immune modulation via natural T-cell—A-007 interactions, the further polarization provided by an external programmed magnetic field can provide added stimulus to the immune response. Use of such an external magnetic field is referred to as an AMTR (A-007 Magnetic Transistor Resonator).

Some of the disclosed methods therefore concern the use of various resonance modulators, such as hydrazones, that are able to function as quantum chemical modulators/sensors/transmitters of immune profiles. These agents are therefore useful in documenting and treating changes in epithelial surfaces (such as the skin) that are connected via biological networks with lymphatic circuits. Peripheral modulation of lymphocytes and precursor cells in the skin is an early warning network for systemic lymphocytes and a natural mechanism by which living systems detect foreign changes and objects (viruses, bacteria, chemicals, cancer cells, etc). Invasion by cancer and foreign chemical/biological objects can produce electrical and emergent behavioral changes that require monitoring/alterations to insure health. Hence the ability to monitor the status of this peripheral modulation provides an important new medical diagnostic and therapeutic tool.

### EXAMPLE 2

### Anti-Neoplastic and Immune Modulating Characteristics of 4,4′-Dihydroxybenzophenone-2,4-dinitrophenylhydrazone (A-007)

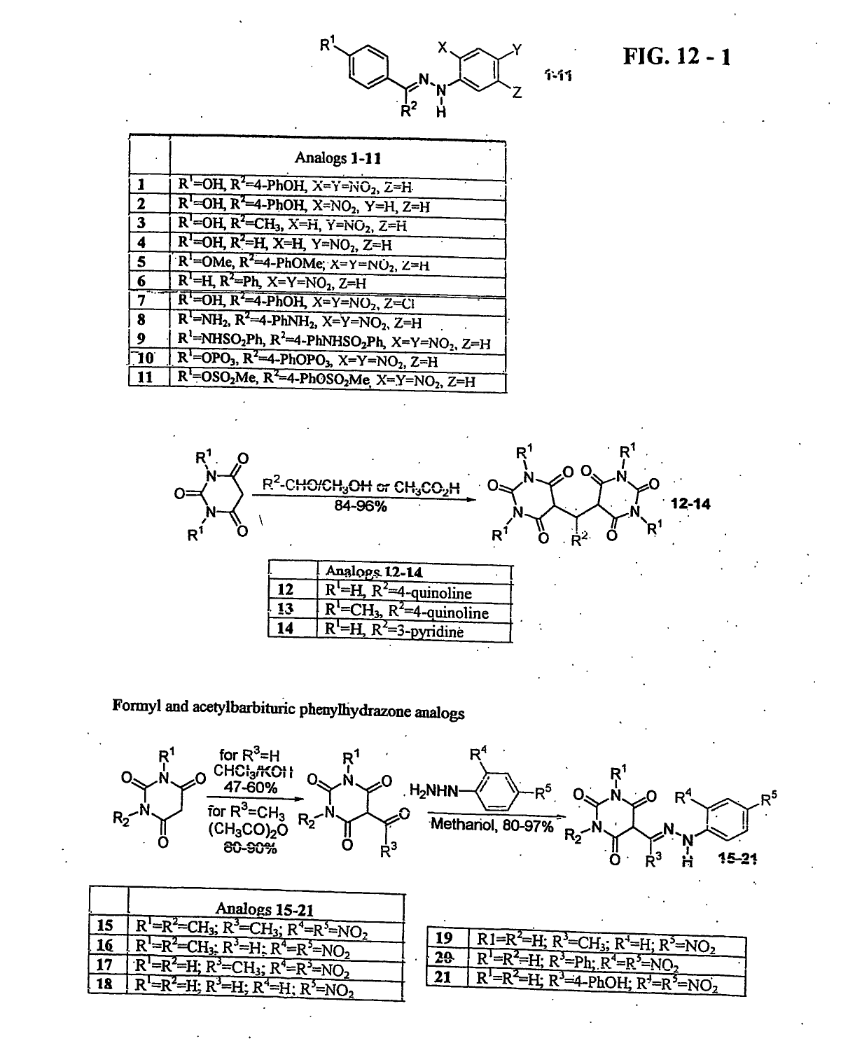

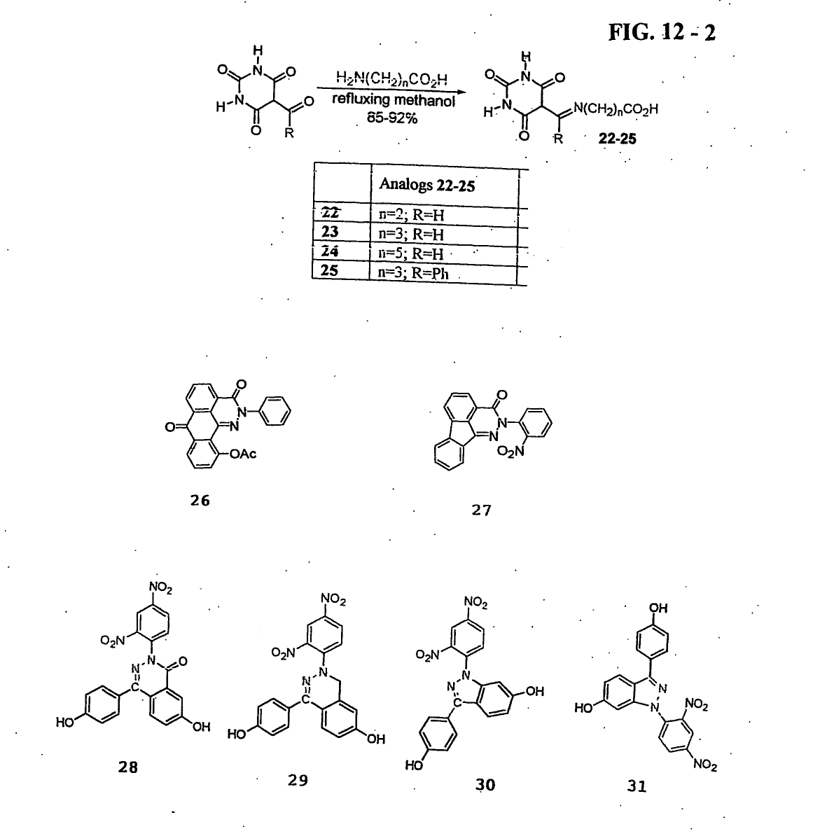

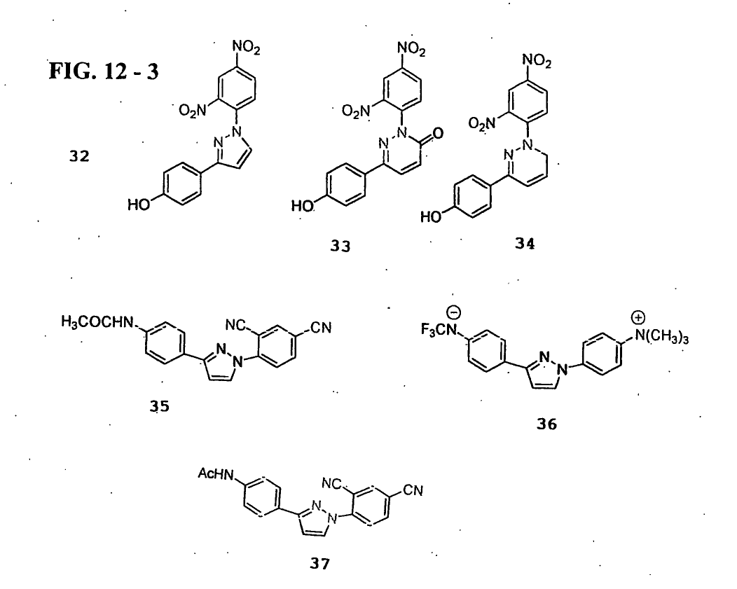

4,4′-Dihydroxybenzophenone-2,4-dinitrophenylhydrazone (A-007, compound 1 in FIG. 12), has produced significant anticancer activities in Phase I/II anticancer trials (IND 47,470). Among fifty-three (53) people treated with topical A-007 (as a 0.25% gel), 37% objective remissions have been observed with ten complete responses.

TABLE 1
Responses of Cutaneous Metastases to A-007 0.25% Gel
Applied Topically to Cancer Lesions Twice Daily

| Cancer | # | Applied (g) | M/F # | Response | Duration (wks) | Toxicities |
|---|---|---|---|---|---|---|
| Breast | 27 | 0.01-2.43 | 30 F | 5 CR, 7 PR, | 6 wks-14.5 mos | 7 skin (mild) |
| | | | | 6 stable | | |
| Melanoma | 5 | 0.02-0.50 | 3F/2M | 1 PR, 3 NR | 3-15 | 2 Skin (mild) |
| KS | 4 | 0.02-0.22 | 1F/3M | 2 PR, 2 NR | 11 | 0 |
| H/N (Sq. Cell) | 1 | 0.091 | 1M | NR | 0 | 0 |
| NHL | 4 | 0.02-0.16 | 1F/3M | 1 PR | 3.5 yrs | 0 |
| Angio- | 2 | 0.02-0.06 | 1F/1M | 2 NR | 0 | 0 |
| sarcoma | | | | | | |
| Anogenital | 10 | 0.05 | 9F/1M | 5 CR, 2 PR | 1.8+ yrs | 0 |

Note:
KS = Kaposi's sarcoma; H/N = head/neck; NHL = non-Hodgkins' lymphoma; CR = complete response; PR = partial response >50%; NR = no response). All patients treated had received at least 2-regimens of systemic therapy (local surgery for the anogenital cancers) prior to initiating A-007.

It has now been determined that A-007 does not act via a cytotoxic mechanism, as evidenced by a substantial absence of A-007 induced local or systemic toxicity. Histochemical assays, from biopsies of human skin topically treated with A-007, confirmed that increased infiltrates of a variety of T-lymphocytes (CD4+, CD3+, CD8+, and CD45+) had occurred during treatment. Increased skin infiltrates of CD11c+ dendritic cells were also observed in treated areas. Immunohistochemical (IHC) studies to date have found that immune modulation had occurred in vitro and in vivo following exposure to A-007. Hence the resonance modulators disclosed herein can have a therapeutic effect without diffuse cytotoxicity that often causes collateral clinical harm.

X-ray crystallography data revealed that A-007 (as monoclinic crystals) exists as two unique molecules, which differ only in the orientation within the bis-diphenylmethane group, where the rings are approximately perpendicular to each other (and rotated approximately 90° from the orientation of the rings in each rotamer) as shown in the accompanying figure. Both rotamers showed strong intramolecular hydrogen bonds between the —NH of the —HN—N═C— moiety and an oxygen of the o-nitro group. Thus, there are at least three unique moieties present in A-007 that may contribute to its overall biological activity—a dihydroxy-diphenylmethane, a hydrazone and a dinitropheyl moiety. However, despite A-007's high electrophilicity, it substantially lacks chemical reactivity under physiological conditions in the human body. For example, the NH on the hydrazone group would be expected to undergo methylation or acylation, but that is substantially absent when A-007 is administered to a human subject The drug is excreted substantially unchanged. Furthermore, A-007 is very insoluble in body fluids and is not absorbed into the blood. A-007 has not been detected in the blood from any of the subjects who have been treated with topical A-007. An effective assay for plasma A-007 has been developed and verified its stability.

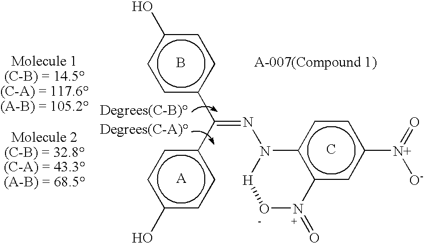

X-Ray Crystallography Characteristics of A-007

### EXAMPLE 3

### Resonance Modulators and the Immune System

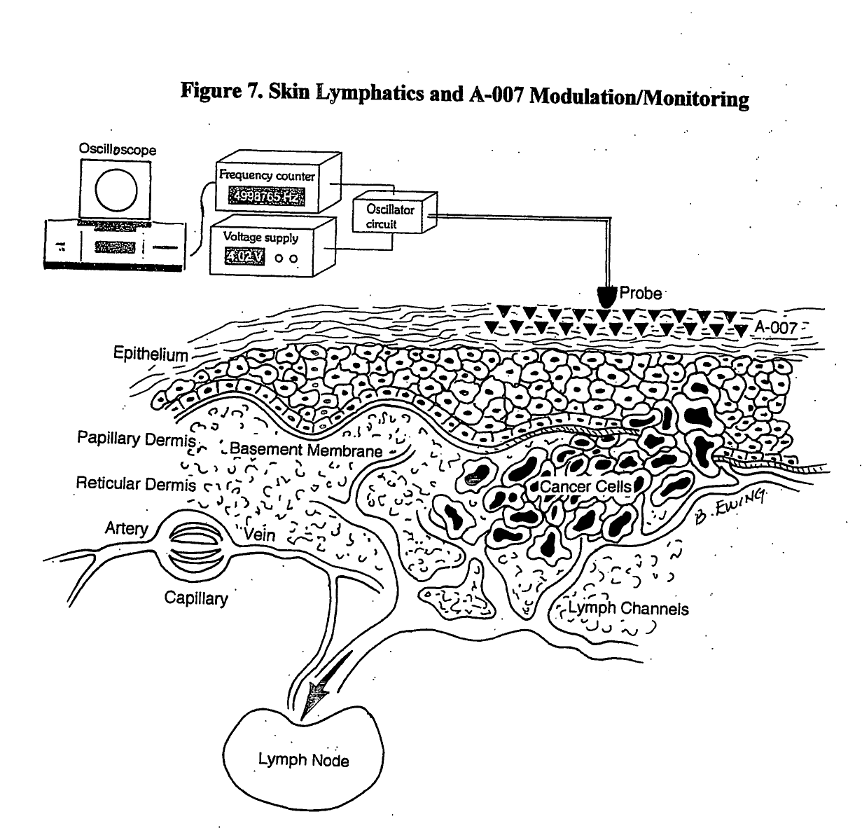

As shown in FIG. 7, the lymphatic system includes lymphatic vessels that communicate with other structures and organs that contain lymphatic tissue in a specialized form of reticular connective tissue. Lymphatic vessels include lymph capillaries, which combine into larger lymph vessels (lymphatics) that resemble veins in structure, but have thinner walls and more valves. Lymph nodes are distributed throughout the body, with the most intense concentration in the face and neck, axillae, thoracic cavity, intestines, groin, elbows and knees. Shallow lymphatic channels of the skin generally follow veins, while deeper lymphatics generally follow arteries. These lymphatics function to network lymph fluid throughout the body, but they are also an important distributed aspect of the immune system that functions in surveillance and defense against foreign cells, such as microbes and cancer cells.

The lymphatic system contains numerous types of lymphocytes. Some of these lymphocytes are T-cells that destroy foreign cells directly or indirectly by releasing cytotoxic substances. Other lymphocytes are B-cells that differentiate into plasma cells that secrete antibodies against antigens to help eliminate them. The lymph nodes filter foreign material carried by the lymph fluid, so that segregated foreign material can then be destroyed by phagocytosis. The spleen, thymus and tonsils are other lymphatic organs that produce B-cells, T-cells, and other lymphocytes.

A skin-associated portion of the immune system is illustrated in FIG. 7, in which tumor cells are shown in the dermis and epidermis. Dendritic cells from the lymph system interact with the tumor cells, and recruit other immune effector cells (such as T-lymphocytes) from lymphatics.

CD45+ surface receptors are present on lymphoendothelial cells, and in particular on dendritic cells. Dendritic cells (DC) are antigen-presenting cells (APCs) involved in the initiation of the immune response. Serving as immune system sentinels, DCs are responsible for antigen (An) acquisition and subsequent transport to T-lymphocyte rich areas. The DCs are present in lymphatic tissues, such as peripheral cutaneous tissue, as well as in lymphoid organs. Once immature DCs interact with an antigen and become activated, the mature APCs are capable of specific immune responses. Secondary lymphoid organs, such as the skin, recruit both naïve T-lymphocytes and An-stimulated DCs (APC) into T-cell rich lymphoid zones/networks (nodes, etc). Co-localizing these early immune responses constitutes cognitive T-cell activation.

Both cancer and chemicals can produce emergent behavior in healthy skin with collective distributed intelligence of lymphocyte populations. Effective recognition responses require both DC (APCs) and lymphocyte cytokine effectors. Because chemicals and tumor cells often have limited expression of microhistochemical (MHC) antigens and lack co-stimulatory molecules, they are not effective modulators of APCs. One mechanism for the development and progression of cancer and allied diseases is lack of MHC antigenic properties that would otherwise produce emergent behavior in lymphatic networks. Similarly, life-threatening chemical and/or biological contacts can also induce emergent lymphatic behavioral patterns. A-007 represents a simple “organic” molecule, that is sufficiently electrically endowed to act as a hapten/An, and/or through electromagnetic field effects (EFE), to modulate or up-regulate emergent lymphocyte networks. It represents a new class of renaissance molecules referred to herein as resonance modulators. It is believed that up-regulation of the CD45+ receptor is one initiation site for the A-007-induced immune modulations that are observed in patients with cancer.

CD45+ is expressed on dendritic cells, lymphocytes, monocytes, and leukocytes, as well as some neoplastic cells, as a protein tyrosine phosphatase (TP), which together with other members of the PTPs, are responsible for phosphorylating tyrosine residues. Blockade of the CD45+ receptor sites with anti-CD45 antibodies inhibits T-cell activation and prevents mitogen (lectin) activation of naïve T-cells. CD45+ receptor surfaces contain arginine, serine/threonine and cysteine moieties, which can bind to and/or transfer natural ligands to the surface of APCs, as well as hydrolyze tyrosyl phosphates. A-007 does not inhibit or block CD45+, but up-regulates CD45+ lymphocytes and dendritic cells (to APCs) via electrostatic/non-covalent binding with Arg, Cys, Ser/ Threo, etc as illustrated in FIG. 1. Different resonance states of A-007 are capable of interacting with different immune cells.

A-007-activated DCs are capable of initiating mitotic events with naïve human blood peripheral mononuclear cells (PBMC) and up-regulating both CD45+ and CD11c+ receptors in human peripheral dendritic cells. Dendritic cells in cancer tissue are up-regulated from CD45RA+ to CD45RO+/CD45RB+ following exposure to topical A-007 during topical treatment of skin lesions. Thus, A-007 is not an inhibitor of CD45+, but an up-regulator or modulator of the molecular sites. These properties allow a crystal or implanted skin pellet of A-007 to increase both local and more remote immune cell populations (such as splenic dendritic cell populations), and increase concentrations of immune effector cells, such as CD8+ cytotoxic lymphocytes (CTL). The influence that functional group substitutions may have on A-007's intra-/intermolecular hydrogen bonding and electrostatic interactions is presented below.

This evidence demonstrates that A-007 has the ability to interact peripherally in the skin and other epithelial surfaces with dendritic cells to induce maturation of the antigen presenting cell (APC). In addition, at least one cell surface receptor (CD45+) appears to be up-regulated by A-007. It is believed that this initial interaction is via dendritic and other lymphocyte precursors, such as antigen presenting cells. Through this chemical interaction, APCs and other early recognition cells are capable of being sensed and modulated with maturation of the lymphocyte recognition cascade—CD4+, CD8+, etc. and associated cytokines.

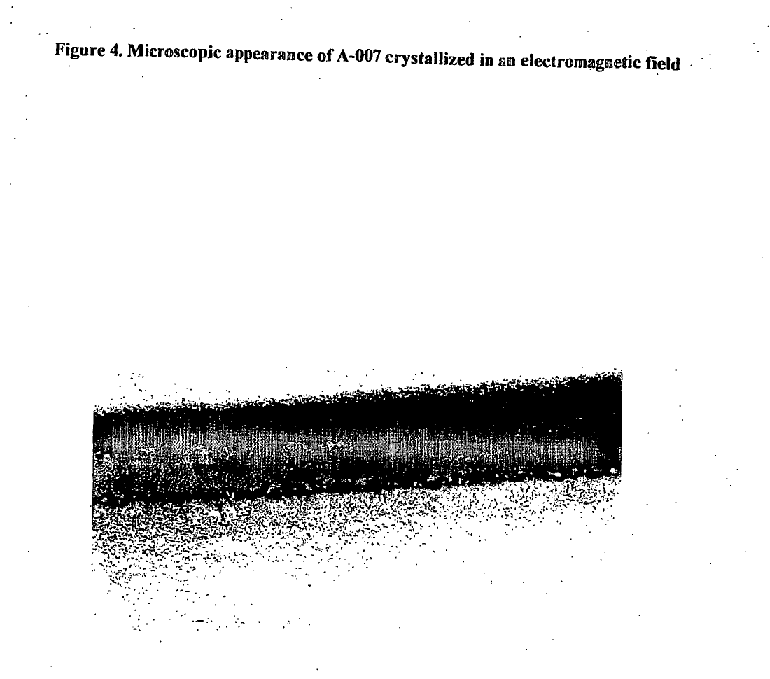

Some of the unusual properties of A-007 that make it suitable as an immune modulator are illustrated by the ability of its crystals to align in an unusual pattern when evaporated on a glass slide between two electrical wires through which a current was introduced. This arrangement of the crystals was obtained by evaporating an alcoholic (5%) solution of A-007 in air on a glass slide between two wires respectively connected to the positive and negative leads of a 9 volt battery. The A-007 condenses in a circumferential pattern at the positive pole, rather than as a diffuse “spot” over the entire field. When evaporation of the alcoholic solution occurs in a magnetic field, a single line of tightly agglutinated crystals forms down the middle of the slide (as shown in FIG. 4). Hence A-007 has electromagnetic properties that interact with induced electromagnetic fields. This characteristic can also be used as a screening test for other resonance modulators.

These unusual electromagnetic properties of the A-007 resonance modulator are further illustrated by the conductance of this compound, as illustrated in FIG. 11. This Figure illustrates the frequency of energy that is measured from the A-007 crystals. These tracings were obtained from several large A-007 crystals, to which two microelectrodes were attached using the magnification of a dissecting microscope. The electrodes were separated by about 1.5-2 mm on the crystal surface and the energy was measured from the contact. The Fluke ScopeMeter was used to record the frequency (Hz) generated. No external applied current was involved, hence the measured frequencies represent a natural physical property of the crystals. These excursions from the baseline frequency are believed to be a translational type of energy that is accumulated from the environment and intermittently released. The baseline frequency is likely from ambient electromagnetic radiation. Excursions from the baseline frequency of at least 30-40 Hz is generated by the resonance of the crystals, which is illustrated in FIG. 11 by the intermittent excursions (indicated by the peaks in frequency) from the baseline frequency of about 60 Hz. This ability to emit natural frequencies of energy (for example at a frequency of at least 20 Hz from baseline, for example to between 30-40 Hz), and/or at least once every five minutes (for example at least once every three minutes) is another example of a characteristic of a resonance modulator that allows resonance modulators to be screened and selected for further testing.

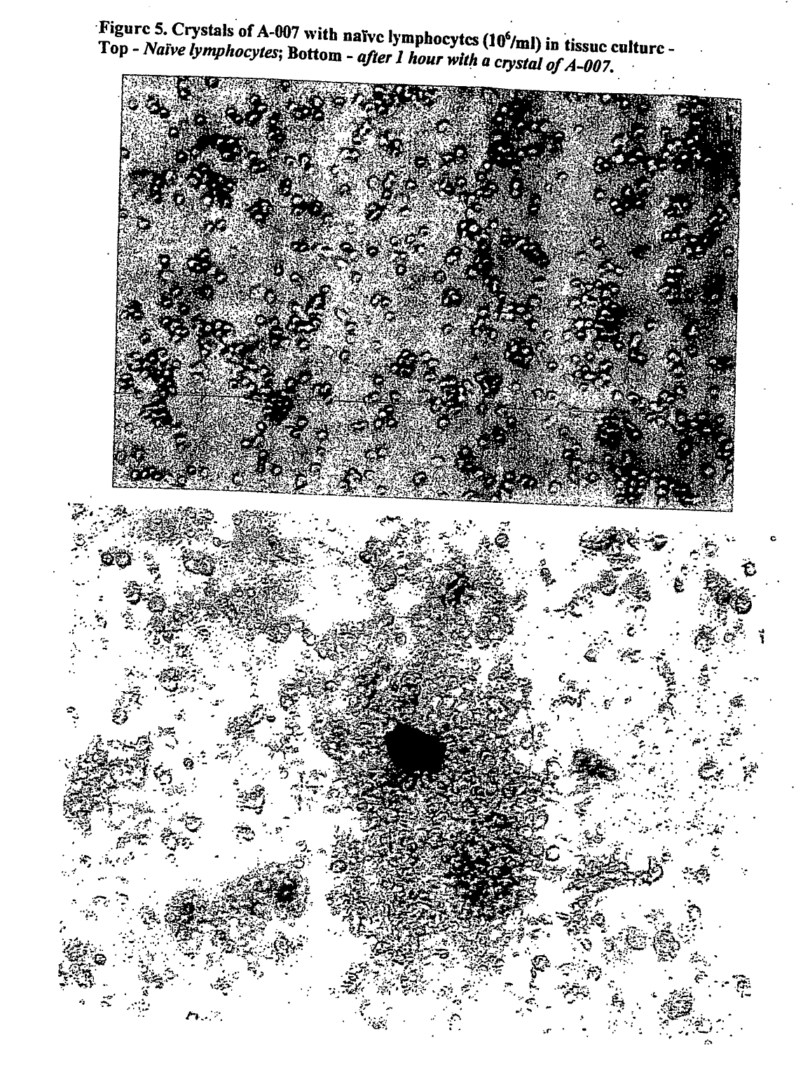

When a crystal or a pressure pressed pellet of the chemical is submersed into a tissue culture media with naive human lymphocytes (obtained from peripheral blood), lymphocytes and dendritic cells aggregate around the crystal or pellet, as shown in FIG. 5. Crystals (0.5 mg) of A-007 were placed into RPMI media containing 5% bovine serum albumin, antibiotic preservatives penicillin/streptomycin, and naive lymphocytes obtained from the buffy coat of blood from a healthy person. The top photograph in FIG. 5 illustrates the general dispersion of naïve lymphocytes in a diffuse cellular pattern in tissue culture before the introduction of the resonance modulator, while the bottom photograph shows the aggregation of the lymphocytes around a crystal of A-007 one hour after the crystal was introduced into the tissue culture. After contact with the crystal of A-007, there is agglutination and increased mitotic activity, as well as colonization of activated dendritic cells into the area of the A-007 resonance modulator agent.

The ability of an agent to induce aggregation of naïve lymphocytes in culture around a test agent is another factor to be considered in determining whether the agent is a resonance modulator suitable for use in the methods disclosed herein. The ability to induce aggregation within one hour is a particularly strong indication that the compound is suitable for further investigation. “Aggregation” refers to a substantial increase in the number of lymphocytes, such as an increase visible by microscopy, as in FIG. 5.

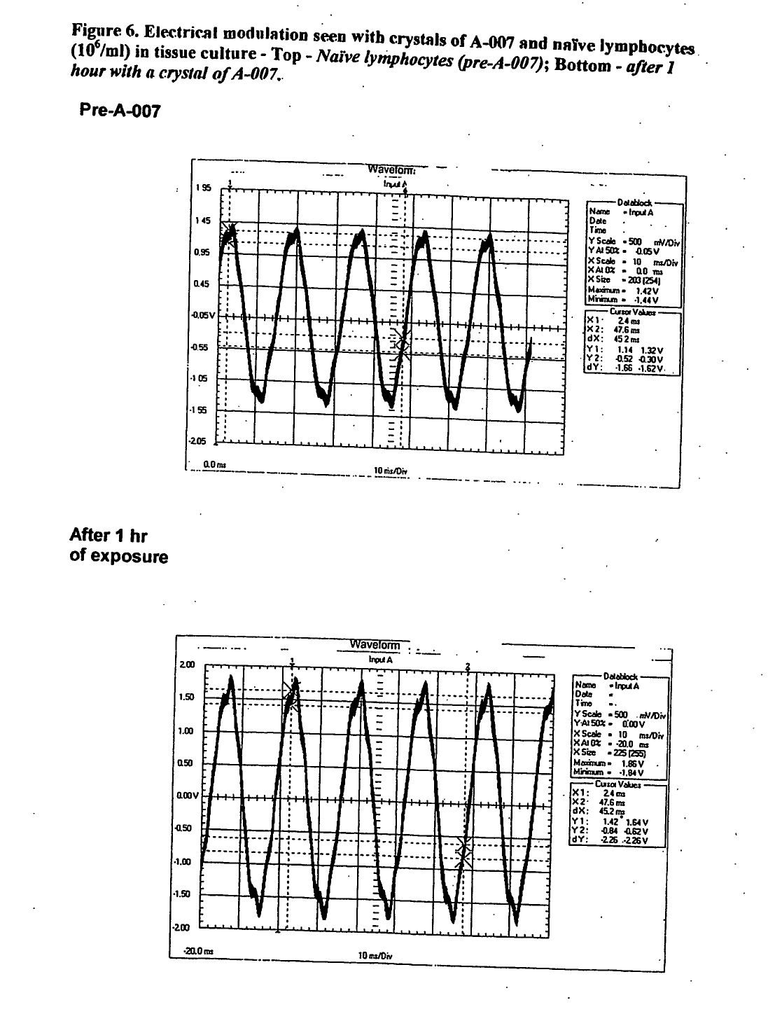

Another unusual electromagnetic characteristic of the resonance modulator is that an increase in amplitude of an electromagnetic wave (such as current) can be measured as lymphocytes mature and agglutinate around the crystal (FIG. 6).

To obtain these waveforms, A-007 was condensed on to sterile glass microscope slides, as shown in FIG. 4. The dried slide was immersed into a petri dish with RPMI (5% bovine albumin), and a pair of sterile microelectrodes were inserted into the media (about 5 cm apart) and placed in contact with the opposite ends of the line of A-007 and the voltage recorded. The A-007 is not soluble in the media and remains on the slide with continuity. The waveform readings of the measured current are provided in FIG. 6. No induction currents were present, hence the measured currents were an inherent characteristic of the resonance modulating compound.

After 1-hour of exposure to A-007, there was an increase in amplitude of the measured voltage over time as the lymphocytes migrate to and attach to the A-007 crystals. This increased amplitude is illustrated in FIG. 6, in which the top waveform was obtained from naïve lymphocytes in culture, and the bottom waveform was obtained from the same lymphocyte culture one hour after the crystal of A-007 was introduced into the culture. Since the crystals do not dissolve, they remain available for continuous interactions. In vitro, the lymphocytes eventually die after about 24 hours from lack of cytokines needed for cellular perpetuation.

Hence another characteristic that can be used to select resonance modulators is an ability to increase the amplitude of alternating voltages in an inherent current produced by the resonance modulator when the compound is placed in culture with naïve lymphocytes.

### EXAMPLE 4

### Resonance Modulators to Monitor Immune Function

As a consequence of their extraordinary properties as resonators, the compounds described herein can be used to monitor electrical activity associated with the immune system. A-007 and other resonance modulators attain significance as an analytical device to measure and control immune characteristics, in which there is a linear relationship between T-lymphocyte and frequency responses. The microbalance that exists is due to the fact that mass sensitivity of a 50 Hz A-007 crystal is approximately 0.057 Hzcm²ng⁻¹, which is approximately 50 times higher than that of an electronic fine-balance with a sensitivity of 0.1 μg. The crystals can be used to measure electromagnetic properties of cellular elements. In particular embodiments, crystals are chosen the have a mass sensitivity of at least about 0.01, 0.03 or 0.05 Hzcm²ng⁻¹. The crystals can be combined with quartz micro-crystals to amplify the electrical interactions (piezoelectric resonator effects) and improve transmission. In particular examples, the quartz microcrystals would be provided in an amount of 10-50% of the total weight of the composition.

The crystals are therefore capable of readily transforming interactions between RPT?+ cell (such as lymphocyte) populations and equivalent electrical circuits of the cutaneous tissues which permit a complete description of the oscillations in the presence of the hydrazones and other resonance modulators. Basically, the resonance modulators serve as a resonator of dipolar movements and electrostatic interactions with cellular elements, such as cells of the tissue and peripheral immune system. The resonance modulator allows these interactions to be monitored for diagnostic purposes, and altered for therapeutic interventions.

The ability to use resonance modulating compounds to monitor immune function permits early diagnosis of a subject's altered ability to recognize a biological insult, such as a toxin or foreign antigen. For example, a subject who has been exposed to a foreign chemical or biological agent may not recognize the exposure and may therefore not respond appropriately. However if the resonance modulator detects a change in function of the immune system, this altered immune status serves as a sentinel event that alerts the subject to a possible unknown toxic exposure. Similarly, subjects exposed to environmental stresses (such as virus/bacteria and traumatic events) may have dendritic cells that do not recognize foreign viruses, chemicals or cancer. Functional impairment of the dendritic cells may allow significant penetration of immune defenses and a threat to life via emergent behavior mechanisms.

The resonance modulators may be used in methods to detect and quantitate changes in cellular profiles of the skin that are associated with normal body immunity and natural surveillance activity. Animal studies and human studies have shown that a resonance modulator such as A-007 is not absorbed from the skin but will attract and peripherally activate populations of dendritic cells, CD8+ cytotoxic lymphocytes and other cellular populations that are needed for the natural modulation toward foreign exposure or irritation. In the event that the body does not respond to the presence of the proposed sensor/stimulant, then aggressive health monitoring and therapies may be pursued.

The monitoring device can take the form of a patch that maintains the resonance modulator in contact with the skin of a subject. The patch can be periodically attached to a voltage meter, for example a voltage meter that provides output in the form of a waveform tracing of the type obtained from an oscilloscope. The amplitude of voltage changes over time is then evaluated to determine how the amplitude changes compare to changes that are observed in healthy subjects. In one example, the amplitude of the voltage waveforms would be expected to increase by a predetermined value (for example at least 10% or 25%) in the presence of the resonance modulator: An amplitude change that is less than the predetermined value is taken as an indicator of a pathologic insult, such as exposure to a toxin or pathogen. This result can indicate the need for more specific testing, such as detection of environmental toxins or pathogens, or imaging tests to detect a tumors or progression of tumors. Alternatively, the abnormal test can prompt the initiation of therapy (or more aggressive therapy), such as the administration of chemotherapeutic agents.

The inherent amplitude and amplitude changes that would be seen would differ for each resonance modulating compound. Hence a specific amplitude change can not be expressed for all resonance modulating compound. Nonetheless, it is the recognition of this characteristic of resonance modulators, and their ability to act as an interface for immune function, that serves as the basis of the disclosed methods. Now that this characteristic has been identified, the specific inherent amplitudes generated by each compound and the changes in amplitude that would be seen in specific disease situations or in generalized immunocompromise, can be determined.

In some embodiments, a piezoelectric crystal may be introduced into the resonance modulator compound to provide maximum A-007 transmission through a transducer effect.

### EXAMPLE 5

### Additional Examples of Resonance Modulators

A variety of resonance modulators are available. In one particular example, the resonance modulator is a polyaryl mononitro- or dinitrophenylhydrazone such as 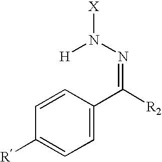
wherein R¹ is hydrogen, hydroxy, 2- or 4-hydroxyphenyl, acetate, nitroso, phosphate, azido, nitrile, amino, dimethylamino, sulfate, methylsulfonate, phosphate, succinate or another water soluble electrophilic group capable of hydrogen bonding; R² is C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; and X is C₆H₃-2,4(NO₂)₂, C₆H₄-4(NO₂), C₆H₄-3(NO₂), or C₆H₃-2,4(NO₂)₂. In particular example, R¹ is OH, R² is C₆H₄ and X is C₆H₃-2,4(NO₂)₂.

In another example, the resonance modulators have the structure: 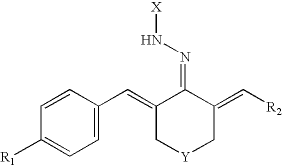
wherein R₁ is hydrogen, hydroxyl, 2- or 4-hydroxyphenyl, acetate, phosphate, azido, nitrile, amino, dimethylamino, sulfate, methylsulfonate, phosphate, succinate or another water soluble electrophilic group capable of hydrogen bonding; R₂ is C₆H₅, C₆H₄OH, C₆H₅, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO (CH₂)ₓCO₂H, or C₆H₅Cl; X is C₆H₃-2,4(NO₂)₂, C₆H₄-4 (NO₂), C₆H₄-3 (NO₂), or C₆H₃-2,4(NO₂)₂; and Y is —O—, —S—, —CH₂—, —N—, —, —CHA- or —CHOA-, wherein A is aryl, ester, amide, lipid, carbohydrate, or peptide residues. In a particular example, R¹ is OH, R² is C₆H₄OH, and X is C₆H₃-2, 4(NO₂)₂.

In another example, the resonance modulators have the structure: 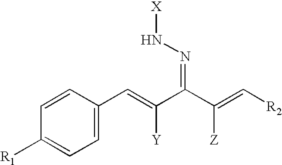
wherein R₁ is hydrogen, hydroxyl, 2- or 4-hydroxyphenyl, acetate, phosphate, azido, nitrile, amino, dimethylamino, sulfate, methylsulfonate, phosphate, succinate or another water soluble electrophilic group capable of hydrogen bonding; R₂ is C₆H₄OH, C₆H₄N₃, C₆H₄CN, CH₃, 4-HO—C₆H₄—C₆H₄, C₆H₄OP0₂0H, C₆H₄OS0₂H, C₆H₄NH₂, C₆H₄NHMe₂, C₆H₄OS0₂Me, C₆H₄OCO(CH₂)ₓCO₂H, or C₆H₅Cl; X is C₆H₃-2, 4(NO₂)₂, C₆H₄-4(NO₂), C₆H₄-3(NO₂), or C₆H₃-2, 4(NO₂)₂; Y is H, (CH)ₓCH₃ (x=0-12), —S—CH₃, nitrile, amino, nitro, azido, succinate, or amide; and Z is H, (CH)ₓCH₃ (x=0-12), —S—CH₃, nitrile, amino, nitro, azido, succinate, or amide. In a particular example, R₁ is H or OH, R₂ is C₆H₄OH or C₆H₅ and X is C₆H₃-2, 4NO₂)₂.

Other examples of resonance modulators are shown in FIG. 12, and include formyl and acetylbarbituric phenylhydrazone analogs (analogs 15-21), formylbarbituric acid Schiff base analogs (analogs 22-25), as well as the other resonance modulators shown as compounds 26-37. Compound 26, for example, is an anthracene. These and other compounds are believed to have immune modulation properties, as well as electromagnetic characteristics that enable them to function as quantum sensors.

A group of suitable resonance modulating compounds is illustrated by the following formula, in which possible substitutions for some of the R groups are shown.

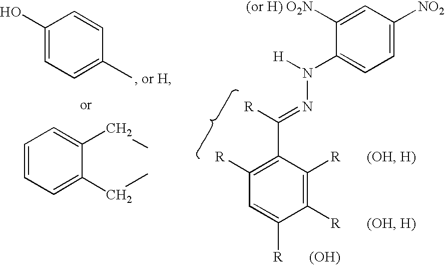

The structures and synthetic methods for many of the described compounds have been previously described in U.S. Pat. No. 4,732,904, which is incorporated by reference. Some compounds that were not described in that patent include 10-[(2,4-dinitrophenyl)hydrazono]-1,2-dihydroxy-10H-anthracen-9-one (DNPA). The synthesis of this compound was performed by dissolving 0.1 mole of 1,2-dihydroxy-9,10-anthraquinone in 50 ml of ethanol and 5 ml sulfiric acid. Gently the solution was heated to 60° C. with stirring and 0.1 mole of 2,4-dinitrophenylhydrazone in 50 ml of ethanol was added slowly with continued stirring. The solution was heated for 2-hrs and cooled in a refrigerator. Deep red crystals of DNPA appeared; m.p. 240-242° C.; yield 92%. The product analyzed for C₂₀H₁₂N₄O₇— found: C, 63.98; H, 3.46; N, 11.13; NMR and mass spectra agreement.

Additional examples of resonance modulators are provided in Example 13.

### EXAMPLE 6

### Preparation of Pellets and Gels

The resonance modulators can be provided in many forms, such as a crystal pellet or gel/cream. The agent can be, for example, implanted subcutaneously or applied to an epithelial surface of the patient whose immune system maybe impaired, or who may have come in contact with a lethal or toxic material capable of down-regulating their immune system The resonance modulator can be incorporated into any form (such as a patch, or a two-dimensional or three-dimensional matrix) that brings it into electromagnetic contact with a target site. “Electromagnetic contact” refers to a sufficient proximity to exert its immune modulating effect as described herein In some examples, the resonance modulator is applied to or over a lesion (such as a tumor or dysplastic epithelium) that is being treated. The resonance modulators can also be chemically modified if desired, for example to form polymers. The resonance modulator monitor is also useful to detect changes in immune function that are associated with cancer, in which there is a loss of auto-modulation of the immune system.

In one example, a pellet of A-007 was prepared by pressing it from 50 mg of pure chemical and sizing it to 16-gauge. Bulk A-007 was prepared using GLP/GMP procedures, with no additives. Depending upon the dissolution properties of the 100% A-007 pellet, additives (stearic acid, povidone, etc) or other pharmaceutically acceptable carriers may be added to it. Pellets may be manufactured, for example, in 25 mg, 50 mg and 75 mg doses. Adjustments in pellet concentrations may be made according to the observed physical properties. (such as dissolution rates) and animal toxicity. Therapeutically effective doses can be determined by known means, and doses to be administered can be varied depending on the condition being treated, or the severity of a disease.

The resonance modulators can also be provided in the form of a gel, such as a preparation of propylene glycol/methyl cellulose, for application to a target area (such as a portion of the body) in which it is desired to focus an immune response. The gels may be provided in a tube, such as 50 mg of the active agent suspended in the gel. These gels are particularly convenient for application to the skin or other epithelial surface (for example skin of the chest wall, back, or anogenital area). In some embodiments, the gel is dispensed from an elongated applicator tube (such as a rectal or vaginal applicator), for example to apply it rectally or intravaginally.

Alternatively, the pellet can be inserted into a target region, such as the chest wall. In other examples, the agent is suspended in tetrahydrofuran (THF) or another non-toxic aerosol, and introduced into to the tracheobronchial or oropharyngeal area to treat cancer of the oral cavity or upper respiratory tract.

It has been found that the pellet stimulates the deposition of uric acid crystals, which can result in painful nodules. Application to the surface of the skin is therefore more preferred than insertion into the skin, but either approach will produce the immune modulation described herein.

### EXAMPLE 7

### External Monitors of Immune Activity

As previously noted, the described agents can both monitor and/or up-regulate immune profiles in the presence of emergent behavioral alterations that are associated with pathological conditions.

In its simplest form, the monitor is a device for detecting changes in electromagnetic properties of the resonance modulator as it interacts with a biological system. The interaction can be observed either in vitro or in vivo. Hence the monitor can be applied to a subject, or to biological samples taken from the subject (such as a tissue culture), for further analysis.

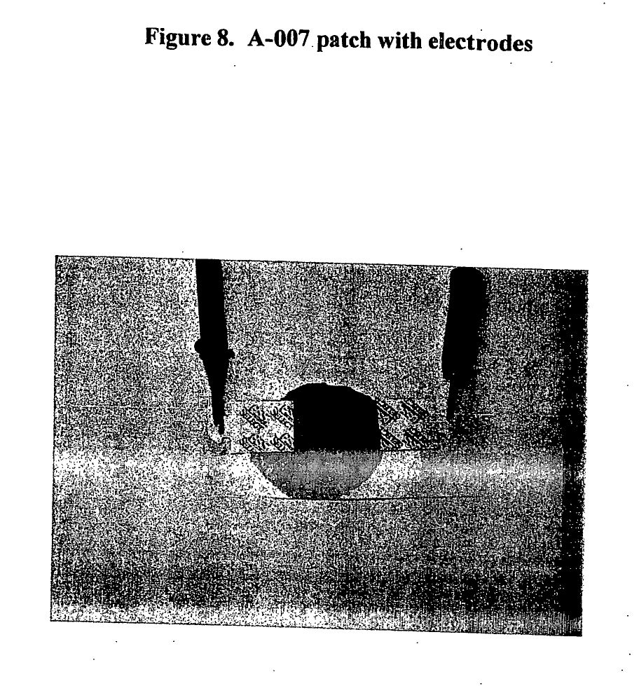

An example of a monitoring sensor is shown in FIG. 8. The resonance modulating agent (A-007) is present on the skin of a patient with lymphoma, as a 0.25% propylene glycolmethyl cellulose gel (0.5 g). Although shown as a gel, it could also be applied as a cream or simple crystals (50 mg) or a pellet (50-100 mg) that can be applied on to or inserted into the epithelial surface or the skin (or other surface) with a 16-gauge trochar needle. In the particular embodiment shown in FIG. 8, the agent is applied to the skin as an orange gel, and covered with a 2×3 Tegaderm® patch with two spaced apart attached skin surface electrode tabs of the type used as electrocardiogram leads. The electrodes are positioned on either side of the gel, in a position that allows them to measure a voltage potential across the gel. The two probes of a voltage meter are then contacted, one probe to each electrode tab. The voltage meter preferably is associated with an oscilloscope that measures changes in voltage over time, so that the amplitude of the detected voltage over time can be seen. In particular embodiments, the voltage meter is a Fluke® Scopemeter 192/196/199 having software that permits the voltage waveforms to be filtered to remove background voltages, and to be viewed, recorded, measured and analyzed on a computer. Changes in the amplitude of voltage over time are then observed to monitor immune response to the resonance modulator.

After establishing a baseline amplitude and/or frequency at the time the resonance modulator is applied to the skin, the electrodes are then subsequently attached to the oscilloscope to monitor amplitude and frequency of electrical transmission from the test site. For a resonance modulator such as A-007, the amplitude of the voltage changes over time will increase.

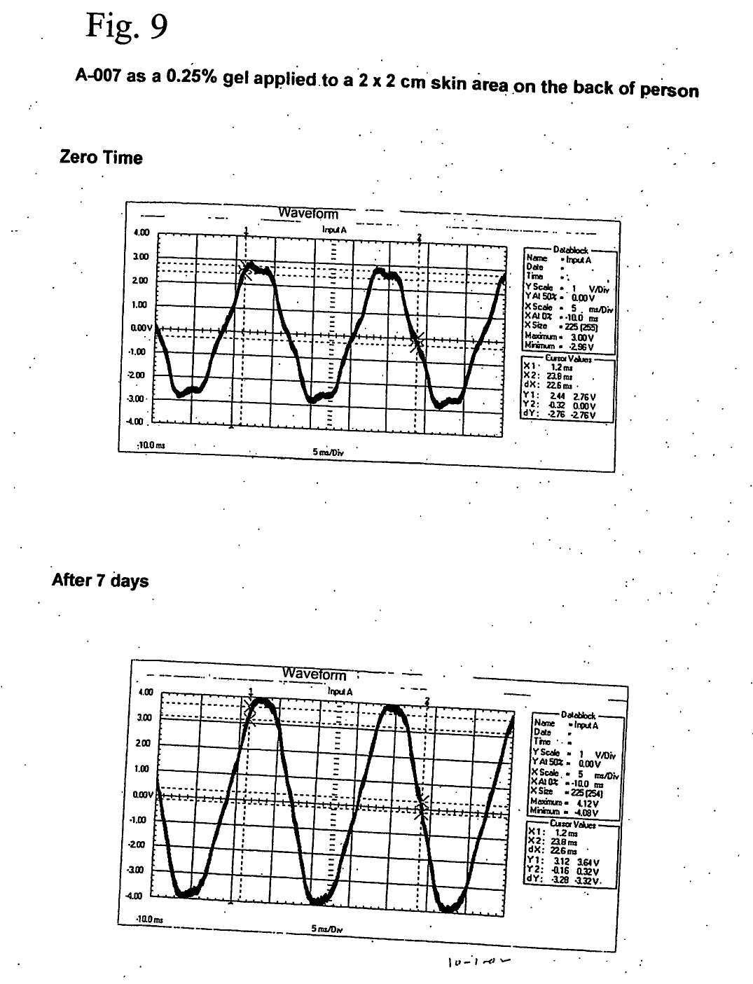

The observed amplitude changes in a normal test subject are illustrated in FIG. 9, in which the top panel shows the baseline measurement at time zero, in which the amplitude (as measured from zero) of the waveform is about 3 volts. After the patch was in place seven days, the tips of the voltmeter probes were again attached to the electrode leads, and the changes in voltage over time were recorded, as shown in the bottom panel of FIG. 9. A measurement was made of the electrical activity associated with agglutination and increased mitotic activity brought about by migration of activated dendritic cells into the target area of the patch. FIG. 9 shows that the amplitude of the waveform (as measured from zero) increased by about 25%, from 3 volts to 4 volts.

Although this example has illustrated measurement of changes in electrical activity with a voltmeter, electrodes could also be sensed with an external laser beam sensor that detects changes in local chemical—cellular interactions. Since the chemical is capable of conducting electrical energy via resonance, the chemical will provide signals of stages of cellular interaction associations. For example, a molecular scanner such as a laser could be used to excite the chemical or assay at its absorption wavelength, which is 404 nm for A-007. One such molecular scanner is an Edmund Industrial Optics solid state tunable laser (wave length 425 nm) that is programmed to record or generate specific excitations associated with A-007 resonance and cellular interactions. These high-performance lasers may be programmed to recognize A-007 spectral characteristics on a nano scale much like a bar code scanner.

### EXAMPLE 8

### Enhancement of Resonance Modulation

The resonance modulators also provide a relatively simple method to enhance immune function, for example in subjects suffering from impaired immunity. Such subjects may, for example, have an immunodeficiency disease (such as HIV/AIDS) or a toxin induced immune defect (such as leucopenia induced by an antineoplastic chemotherapy drug or an environmental toxin). Other subjects may have an impaired immunity of the kind often seen in subjects with malignant tumors. The resonance modulating agent acts as a coupling agent or interface with the immune system that allows the immune system to be not only monitored, but also therapeutically munipulated.

As already shown, a targeted immune response can be directed to a target region (such as the vicinity of a tumor) by introducing the resonance modulator into or adjacent the target area. Exposing the resonance modulator to an electromagnetic field that stimulates the electromagnetic properties of the agent then stimulates the resonance modulator. For example, the agent can be exposed to an external magnetic or electrical field. One example of such an electromagnetic field is one that signals or tunes the frequency that would provide the highest concentration of a resonant form of the resonance modulator (such as one of the forms of A-007 shown in FIG. 3) to provide maximum interface with cellular receptor surfaces. In this example, a laser would be chosen to apply an electromagnetic field in the form of laser energy that possesses similar wave lengths (400-450) to those demonstrated by A-007. Although A-007 does not have lasing properties, a programmed laser could recognize its spectra characteristics. A simple magnetic field generated by 2 AA or 3 V batteries could non-specifically activate a surface gel of A-007 or 9 V for a subcutaneous pellet. A more specific tunable laser would be able to scan the area and be more specific about the optimal electromagnetic field to apply. Selecting a desired frequency will increase the amplitude of the voltage changes over time seen in the resonance modulator, and in turn enhance or activate an immune response.

The device for exposing a resonance modulating agent to an external stimulation is illustrated in FIG. 7, in which A-007 is shown applied to the surface of the skin over a target area that contains a tumor that is present in both the dermis and epidermis. Although the A-007 will attract dendritic cells and lymphocytes into the target area, tumors can often evade normal immune surveillance, and it is helpful to further stimulate the immune response by heightening activation of the resonance modulator. A magnetic probe shown positioned over the A-007 that has been applied to the skin can provide such heightened activation. The magnetic probe is connected to a device that includes an oscillator circuit, a frequency counter, a voltage supply, and an oscilloscope. The device is activated to apply a magnetic pulse to the A-007 to enhance its resonance modulating properties.

An example of a suitable magnetic pulse generator is The Magnetic Pulser, which is a High Intensity Momentary Time-Variant Pulsed DC Magnetic Field Therapy Generator MODEL #: MPG5 available from Health Canada. The Magnetic Pulser (MPG5) is designed to generate an intense (˜43,133 Gauss), momentary (˜2.5 mS) pulsed DC magnetic field that can be used to stimulate the resonance modulator compound. The magnetic pulse can be applied for a sustained period of time, sufficient to enhance the activity of the compound. For example, the pulsed DC magnetic field could be applied for 5-60 minutes or longer.

A simple magnetic field generated by 2 AA or 3 V batteries would activate a surface get of A-007, or a 9 V battery would activate a subcutaneous pellet. A more specific tunable laser would be able to scan the area (such as an area to which the compound has been topically applied) to provide a more specific indication of the electromagnetic field that is to be applied. Selecting a desired frequency of the field applied would maximize the amplitude of the energy produced by the resonance modulator. The duration of time for which the electromagnetic field is applied will depend on the subject's immune status; longer periods of stimulation would be helpful for subject's having a poorer immune status. Although a general magnetic field may be used, a preferred approach is to use a wavelength or energy specific tunable transmitter for wide regions of treatment The effect of the treatment can be assessed by monitoring immune status with easily available modalities, such as peripheral T-cell flow cytometry analysis. These readily available and convenient tests provide additional guidance in the selection of characteristics of an applied electromagnetic field for a particular resonance modulator.

In one example, the applied electromagnetic stimulus is an electric field of the type applied in U.S. Pat. No. 6,190,893, which is incorporated by reference. The device for electrical stimulation includes a gold wire cathode extending along one edge of the target area, and a silver wire anode positioned along an opposing edge of the target area. An EG&G Princeton Applied Research Potentiostat/Galvostat Model 263A (Oakridge, Tenn.) is used as the source of constant potential. The electrical stimulus can be applied for a period of at least one hour, at a steady potential of 100 mV.

Many other devices are available for applying the electromagnetic field to the resonance modulator in a human body or a tissue culture. For example, PCT Publication WO 02/102457 discloses such an apparatus that includes a transducer and a generator to apply an AC signal to the transducer, such that an electromagnetic field is generated with a basic frequency between 0.1 Hz and 4000 Hz. A similar device is also shown in U.S. Pat. No. 5,968,527, which is incorporated by reference.

In some situations it is desirable to use a bioimpedence device to both provide a stimulus to the resonance modulator, and monitor the electrical properties of the target area to which the resonance modulator has been applied. Direct bioimpedence measuring systems use a current generator to generate a continuous, constant amplitude and frequency current through a human or animal body segment, for the purpose of measuring tissue conductance. Frequencies in the range of 30 KHz-30 MHz have typically been used. Impedence to the continuous current flow in the body segment generates a voltage difference across the body segment, and a bioimpedence meter measures the impedence in the body segment. Examples of bioimpedence devices are shown in WO 01/76475; U.S. Pat. No. 4,805,621; and U.S. Pat. No. 5,529,072. The bioimpedence probe is placed over the target area to which the resonance modulator has been applied, and the voltage difference is introduced across the target area. Changes in bioimpedence are also used to monitor the status of the subject's immune system. It is believed that an increased bioimpedence would be seen as immune status improves.

### EXAMPLE 9

### Use of the Resonance Modulator in Treatment of Lymphoma

An A-007 0.25% gel (0.5 g) was applied to the skin of a 42 year old patient with lymphoma, as described in Example 7, and there was improvement in the patient's peripheral lymphocyte profile (as measured by a total lymphocyte count) as well as an increase in amplitude of the sigma waves, indicating that there was increased activity in the skin associated with A-007. The increased oscillation amplitude and frequency that occurred over a one-week period of time is reflected in FIG. 9. The sensoring was conducted with a Fluke® ScopeMeter oscilloscope and a PC program (192/196/199) capable of recording and sorting environmental background noise. The 25% increase in voltage amplitude was considered a positive modulation of immunity in this particular example, which was consistent with the improvement in the lymphocyte profile.

### EXAMPLE 10

### Use of Resonance Modulator in Treatment of Cervical Cancer

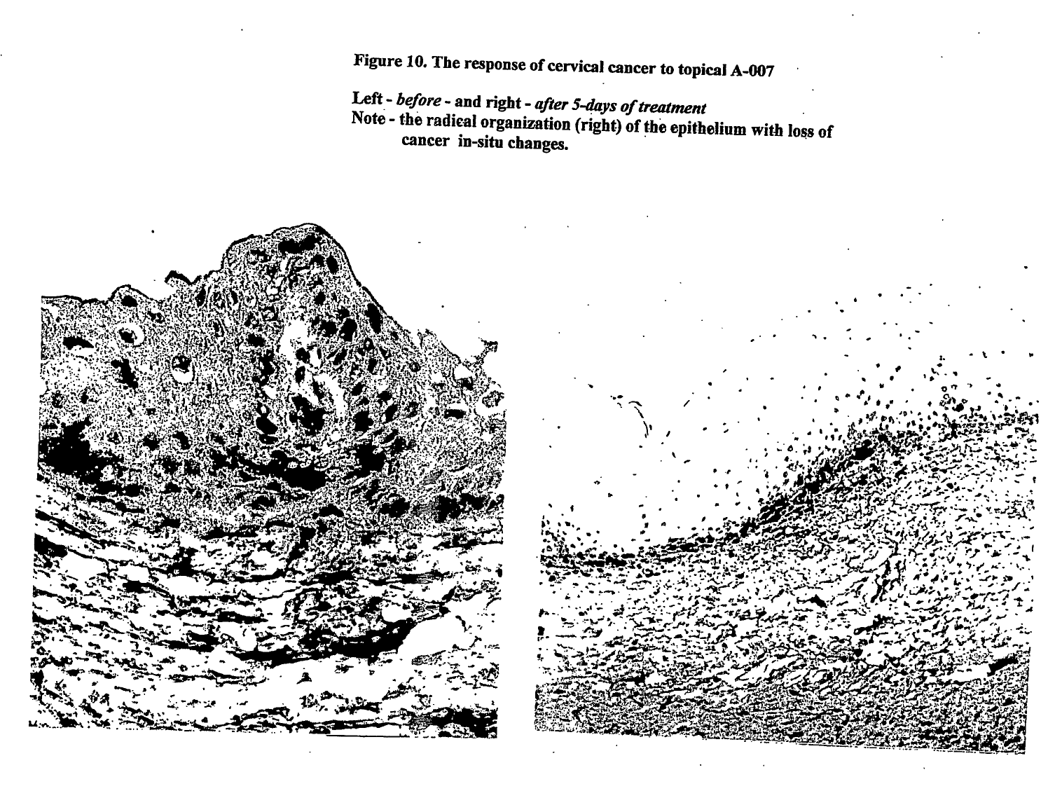

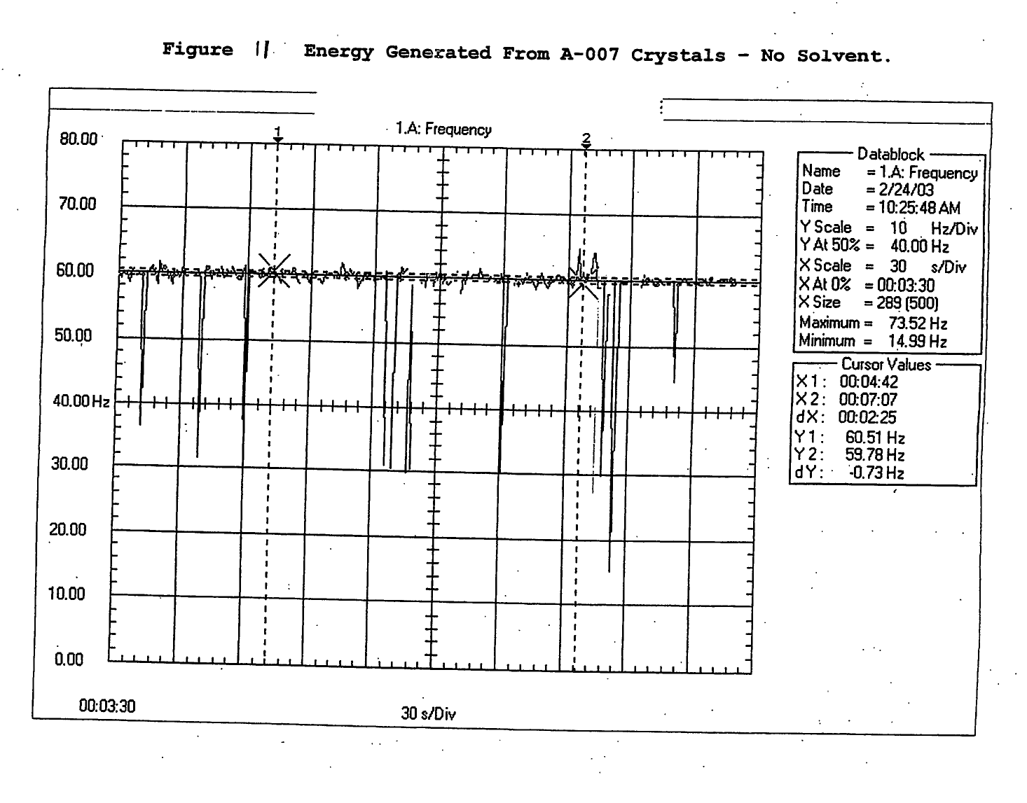

FIG. 10 reflects the changes in the cervix of a 23 year female with early cancer-in-situ who applied the A-007 0.25% gel (2 g) daily for 5-days with a dramatic cure that has lasted for over one year. The photo in the right panel reflects disappearance of the cancer cells in the epidermis and a normal appearing epithelium with influxes of CD45+ T-lymphocytes. The responses are associated with increased organization of T-lymphocyte patterns and disappearance of cancer cells.

### EXAMPLE 11

### Protein Tyrosine Phosphatases

Human protein tyrosine phosphatases “TPs) are a large and diverse family of proteins present in all eukaryotes. Each PTP is composed of at least one conserved domain characterized by an 11-residue sequence motif containing cysteine and arginine residues, the latter are known to be essential for catalytic activities. The sequences of PTP share no similarity to serine or threonine, acid or alkaline phosphatases. The diversity in structure within the PTP family results primarily from the variety of non-catalytic sequences attached to the NH₂— or COOH— termini of the catalytic domain. There are numerous PTPs involved in intracellular phosphate metabolism and domains. The diversity of the extra cellular segments presumably reflects the variety of ligands to which the PTPs are exposed and catalyze phosphate transfer.

The extracellular PTPs are one class of PTP receptors that are related to surface recognition and adhesion molecules of leukocyte cell surface recognition. The PTPs are not only associated with human cells, but also present in prokaryotes and viruses, and bacteria. In the pathogenic bacterium Yersinia, the causative agent of bubonic plague, the Yop2b tyrosine-specific PTP is an essential virulence determinant.

Numerous studies have demonstrated the importance of PTPs in physiological processes. Phenotypic defects and hyperproliferative behavior of T-and B-lymphocytes, granulocytes and macrophages are considered to be key issues in the development of cancer and autoimmune diseases.

The catalytic domain for the PTPs has been described in crystallographic studies, as reviewed in Z. Jia, et al., Structural Basis for Phosphotyrosine Peptide Recognition by Protein Tyrosine Phosphatase 1B, Science 268: 1754-1758, 1995. This reference and Z. Xu et al., Negative regulation of CD45 by differential homodimerization of the alternatively splied isoforms, Nature Immunology 3:764-771, 2002, disclose the various types of cells, bacteria and viruses that express PTPs. The present example reviews the interactions and results for aryl hydrazones as PTP modulators.

A review of 4,4′-dihydroxybenzophenone-2,4-dinitrophenyIhydrazone (A-007) and its structure property relationship (FIGS. 1 and 3) reveals that it does not satisfy Lipinsid's “Rule of 5”. The “Rule of 5” predicts that for absorption or permeation, a drug is more likely to have less than five hydrogen-bond donors, less than ten hydrogen bond receptors, the molecular weight is less than 500 and/or the calculated log P is less than 5 (CAChe Group, Fujitsu, Beaverton, Oreg.). Obviously A-007 and the other hydrazones have greater than 5 hydrogen bond donors as seen by crystallography (Klein, C. L., Gray. D., and Stevens, E. D., Crystal and molecular structures of benzophenone phenylhydrazone derivatives with anticancer activity, Structural Chemistry 4: 377-383, 1993). Thus the external binding to lymphocytes and cancer cells that are described in this patent is in agreement with structural—behavioral observations of the A-007 type aryl hydrazones, in that the drug is substantially not absorbed through the skin to which it is applied. Its effects are therefore achieved over a distance, as would be expected from an electromagnetic effect.

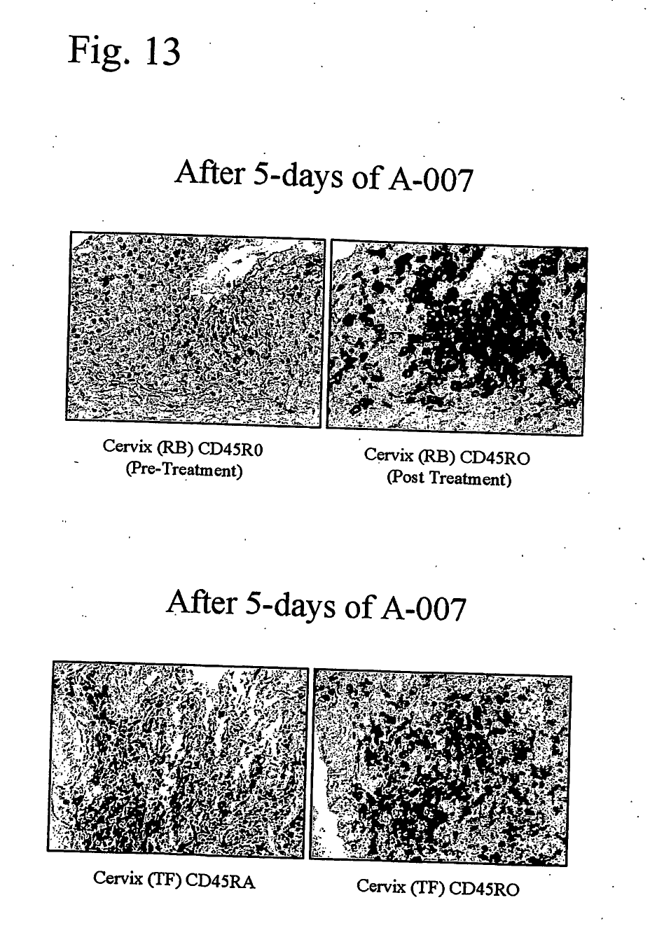

As seen in FIG. 1, A-007 fits well into extracellular catalytic receptors of CD45+ PTP subtype. FIG. 13 confirms that cervical cancer cells (that had been transformed through HPV-induced mutations) undergo cell death in the presence of crystals of A-007; supporting A-007's ability, through its resonance to catalyze HS—SH dimerization of cysteine residues up-grading the receptor and death. This has been verified in patients with cervical cancer (Table 2). A topical gel with 0.25% of A-007 was administered intravaginally in a dose of 2 grams daily for 5 days. A-007 induced cell death was not observed with benign fibroblasts or healthy epithelial cells that did not contain or had not been transformed by HPV. Topical application of resonance modulators such as A-007 to cervical/vaginal epithelial membranes in subjects infected with HPV results in elimination of cancer cells, as well as the elimination of virus particles, and an up-regulation of CD45RO+CD4+ and CD8+ T-lymphocytes. The ultimate therapeutic effect of the topical application of A-007 was induction of cell death in HIV infected cells (Table 2 and FIGS. 10 and 13). As can be seen in Table 2, A-007 increased the presence of T-lymphocytes, and particularly CD45+, CD123+, CD4+ and CD8+ cells. In particular examples, an increase in CD45+, CD4+ and CD8+ cells was particularly noted. In particular, increases of CD45RO+, CD45RA+ and CD45RB+ were noted.

TABLE 2
Effects of A-007 (0.25% Gel) Applied Intravaginally to Humans with Cancer*

| Patient/ | HPV*** | Response | T-Cell Profile | | | Amount A-007 |
|---|---|---|---|---|---|---|
| Histology** | (Response) | (Cancer) | (Tissue/Blood) | | Toxicity | Applied (mg) |
| Vaginal Cancer | No virus | >75% | CD45+ | (↑ 100%) | None | 2.5 |
| (Giant Cell Cancer) | | | CD8+ | (↑ 25%) | | |
| (V-1) | | | CD11C+ | (↑ 50%) | | |
| Vaginal Cancer | +(50%) | CR | CD45+ | NA | None | 2.5 |
| (V-2) | | | CD4+ | (↑ 20%) | | |
| | | | CD8+ | (↑ 25%) | | |
| Cervical Cancer | +(CR) | CR | CD45+ | (↑ 20%) | None | 2.5 |
| (V-3) | | | CD4+ | (↑ 38%) | | |
| | | | CD8+ | (↑ 12%) | | |
| Cervical Cancer⁺ | +(CR) | CR | CD45+ | (↑ 50%) | None | 2.5 |
| (V-7) | | | CD45RO+ | (↑ 50%) | | |
| | | | CD4+ | (↑ 40%) | | |
| Cervical Cancer | +(NC) | CR | CD45+ | (↑ 40%) | None | 5 |
| (V-8) | | | CD45RO+ | (↑ 50%) | | |
| | | | CD8+ | (↑ 50%) | | |
| Cervical Cancer⁺ | +(CR) | CR | CD45RB+ | (↑ 50%) | None | 2.5 |
| (V-9) | | | CD45RO+ | (↑ 80%) | | |
| | | | CD4+ | (↑ 15%) | | |
| | | | CD8+ | (↑ 20%) | | |
| Cervical Cancer | +(CR) | CR | CD45+ | (↑ 20%) | None | 2.5 |
| (V-11) | | | CD45RB+ | (↑ 53%) | | |
| | | | CD123+ | (↑ 21%) | | |
| | | | CD8+ | (↑ 28%) | | |
| Cervical Cancer⁺ | +(CR) | CR | CD45RO+ | (↑ 67%) | None | 5 |
| (V-12) | | | CD4+ | (↑ 55%) | | |
| | | | CD8+ | (↑ 36%) | | |
| Cervical Cancer⁺ | +(CR) | 75% | CD45RB+ | (↑ 72%) | None | 2.5 |
| (V-13) | | | CD45RA+ | (↑ 14%) | | |
| | | | CD4+ | (↑ 9%) | | |
| Cervical Cancer⁺ | +(CR) | CR | CD45+ | (↑ 45%) | None | 2.5 |
| (V-14) | | | CD45RA+ | (↑ 50%) | | |
| | | | CD8+ | (↑ 60%) | | |
| Cervical Cancer⁺ | +(CR) | CR | CD45+ | (↑ 30%) | None | 5.0 |
| (V-15) | | | CD4+ | (↑ 10%) | | |
| | | | CD8+ | (↑ 10%) | | |
| Cervical Cancer⁺ | +(CR) | CR | CD45+ | (↑ 15%) | None | 2.5 |
| (V-16) | | | CD45RO+ | (↑ 31%) | | |
| | | | CD8/4+ | NC | | |
| Cervical Cancer⁺ | +(CR) | CR | CD45+ | (↑ 16%) | None | 2.5 |
| (V-17) | | | CD8+ | (↑ 30%) | | |

Note:
*The gel (0.25%) was administered intravaginally (2 g daily for five days; could be repeated every 3-6 weeks).
**Histology - 1/giant cell and 12/squamous cells;
⁺CIN epithelium present.
***Presence & changes in HP titers.
Note:
patients V-4 to V-6 and V-10 were evaluated but not treated.
NA—not available.
NC—no change.

Although shown as a gel in this example, the A-007 could also be applied as a cream or simple crystals (50 mg) or a pellet (50-100 mg) that can be applied on to or inserted into the epithelial surface or the skin (or other surface) with a 16-gauge trochar needle for short-term treatments.

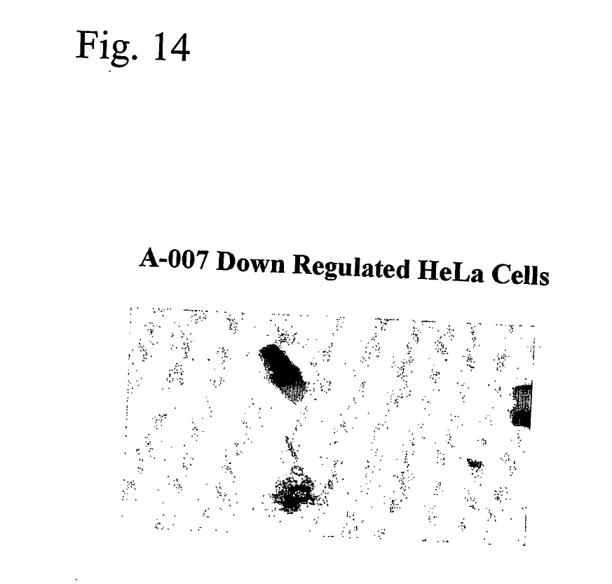

HeLa cancer cells (HPV+) undergo necrobiosis and death when incubated with crystals of A-007 for 24 hours. FIG. 14 shows the clear healthy cancer cells (upper right and lower) migrating to the crystal. Then through cell membrane chemical interphase and modulation of the PTP receptors the cells down regulate, darken with pyknotic changes associated with DNA agglutination resulting in death (lower clumped dark staining cells). The attraction that the crystals have for the cells is associated with intramolecular resonance and associated magnetic patterns described in FIGS. 3 and 4.

Naïve T-lymphocytes are also activated in the presence of A-007 with agglutination of activated lymphocytes (FIG. 5).

Thus, A-007 and the other resonance modulating compounds such as the described hydrazones can play a unique role in not only sensitizing HPV positive epithelial cells to attack by activated T-cells (Table 2) but also has direct effects on HPV positive cells and A-007 resulting in cell and virus death (Table 2). In the latter Table patients were no longer infected with virus after treatments, which illustrates that the agent has anti-viral properties as well. Thus, A-007 has both up-regulation of CD45+ activities in T-lymphocytes, as well as extracellular membrane deregulation and elimination of cells infected with HIV.

These findings suggest that both HPV infected cervical epithelial cells and T-lymphocytes contain extracellular membrane bound PTs with which resonance modulators such as A-007 can interact. The HPV virus may contain an active extracellular PTP receptor, which when exposed to A-007 can undergo a deregulation and death. Regardless of the actual mechanism, the resonance modulators have been found to have effective anti-viral activity against HPV. Moreover, it is believed that T-lymphocytes are activated by a resonance modulator such as A-007 to promote the presence of surface membrane CD45+RO PTPs, which is believed to occur through HS—SH formation within the catalytic receptor site. CD45+RA T-lymphocytes represent resting or naïve cells that are not capable of attacking foreign cells, hence the conversion to CD45+RO markers represents an activation of the cytotoxic arm of the cellular immune system As Table 2 illustrates, the resonance modulator had substantial anti-viral (anti-HPV) and anti-neoplastic activity. FIG. 13 illustrates that the proportion of CD45+RO cells increases substantially after treatment with the resonance modulating agent.

Cells that express PTPs, such as Yersinia pestis pathogens, or PTP+ cells that are infected with pathogens (such as HPV infected epithelial cells) can also agglutinate with A-007. This agglutination is believed to occur by oxidation of cysteine and disulfide bond formation. This bond formation disrupts cell integrity and cell death—releasing virus. In the presence of A-007, viral PTP cell membrane auto-oxidation occurs with viral inactivation. Most of the patients in Table 1 also possessed HPV+ hyperplastic cervical cells containing HPV induced intranuclear changes (CPN) that cleared with A-007 therapy. This further emphasizes that resonance modulators such as A-007 have the ability to destroy benign and hyperplastic cells infected with HPV, as well as malignant cells.

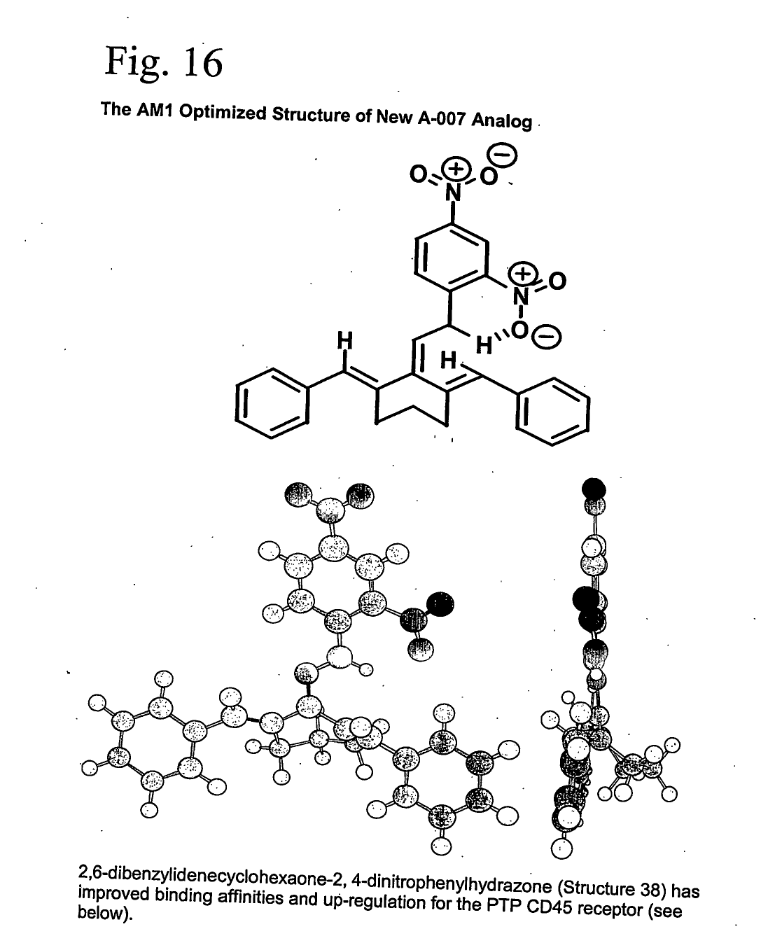

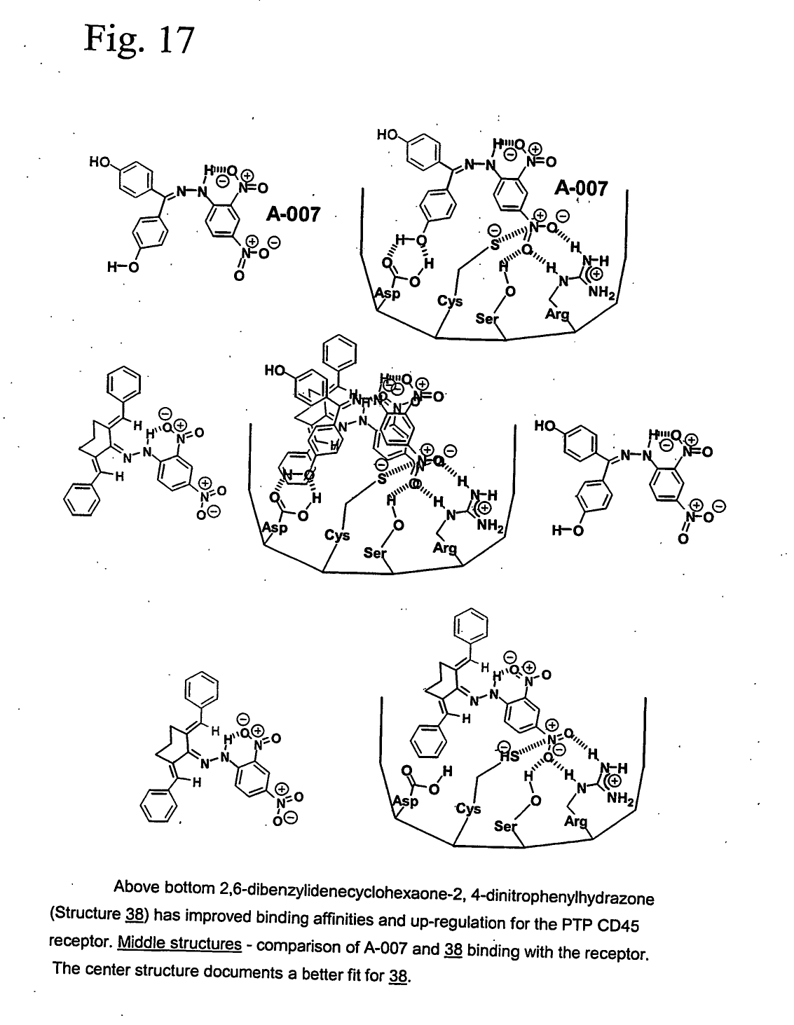

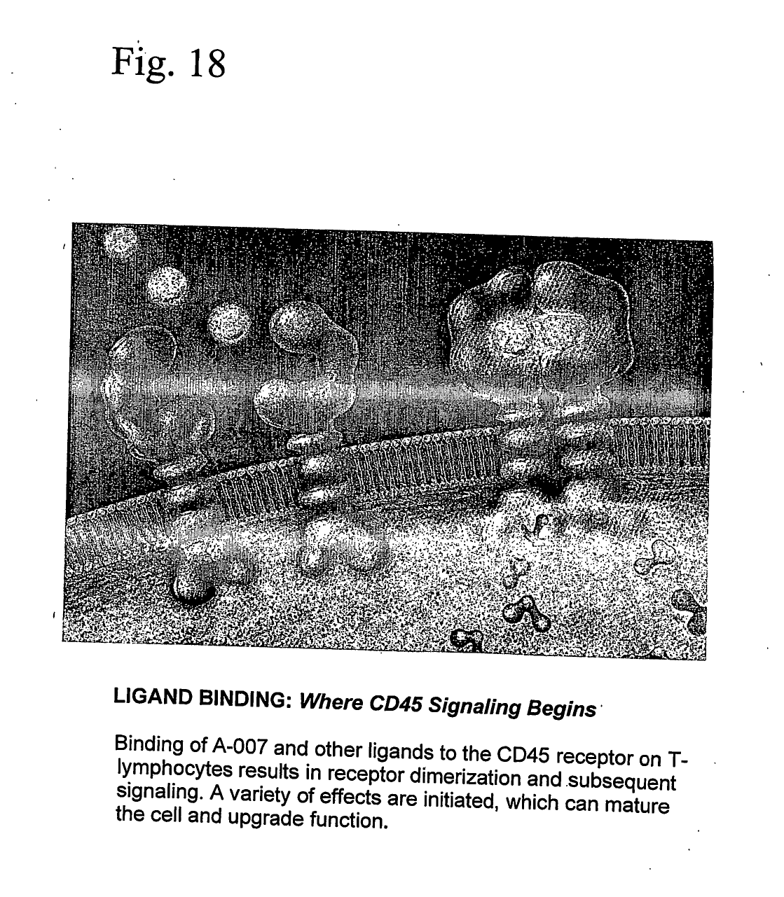

Anticancer and antiviral activities for hydrazone analogs, such as 2,6-dibenzylidenecyclohexaone-2,4-dinitrophenyl hydrazone (Compound 38; see FIG. 16), which has improved binding affinities and up-regulation for the PTP CD45 receptor is also potentiated through exposure to ultraviolet light Ultraviolet light induces Diels-Alder reactions between A-007 and arginine's diimine moiety and adduct formation with cysteine's HS— moiety resulting in up-regulation of the CD45 receptor (FIG. 1). Hence the immune modulating, anti-viral and anti-tumor activities of such resonance modulators can be enhanced by exposing the compound to ultraviolet light.

### EXAMPLE 12

### Anti-Neoplastic and Immune Modulating Characteristics of 4,4′-Dihydroxybenzophenone-2,4-dinitrophenylhydrazone (A-007)

4,4′-Dihydroxybenzophenone-2,4-dinitrophenylhydrazone (A-007, compound 1 in FIG. 12), has produced significant anticancer activities in patients with anal squamous cell cancer (Table 3). A topical gel containing 0.25% of A-007 (2 grams per day of gel) was applied intra-anally daily for five days, and the observed results are shown in Table 3.

TABLE 3
Effects of A-007 (0.25% Gel) Applied Anal to Cancer*

| | | | | | | Amount of |
|---|---|---|---|---|---|---|
| Patient/ | HPV | Response | T-Cell Profile | | | A-007 |
| Histology | (Response) | (Cancer) | (Tissue/Blood) | | Toxicity | Applied (mg) |
| Anal Cancer (A-1) | +(↓ 75%) | 100% | CD45+ | (↑ 10%) | None | 2.5 |
| | | | CD45RO+ | (↑ 25%) | | |
| | | | CD8+ | (↑ 10%) | | |
| Anal Cancer (A-2) | None Detected | 0% | CD45+ | (↓ 20%) | None | 2.5 |
| | | | D45RA+ | (↑ 63%) | | |
| | | | CD45RB+ | (↑ 23%) | | |
| | | | CD4+ | (↑ 13%) | | |
| | | | CD8+ | (↑ 2%) | | |
| | | | CD11C+ | (↑ 15%) | | |
| Anal Cancer (A-3) | None Detected | 0% | CD45+ | (NC) | None | 2.5 |
| | | | All others | NC | | |
| Anal Cancer (A-4) | None Detected | 0% | CD45+ | (↑ 5%) | None | 2.5 |
| | | | CD8+ | (↑ 30%) | | |
| | | | CD45RA+ | (↑ 11%) | | |
| | | | CD45+ | (NC) | | |
| Anal Cancer (A-5) | +(HIV/HPV) | 0% | CD45RA+ | (↓ 10%) | None | 5.0 |
| | NR | | CD45RB+ | (↑ 20%) | | |
| | | | CD4/8 | NC | | |

Notes:
*The gel (0.25%) was administered intraanal (daily for five days; could be repeated every 3-6 weeks prn).
**Histology - squamous cells or adenocarcinoma.
***Changes in HPV titers.
NC—no change.

### EXAMPLE 13

### Further Examples of Resonance Modulators

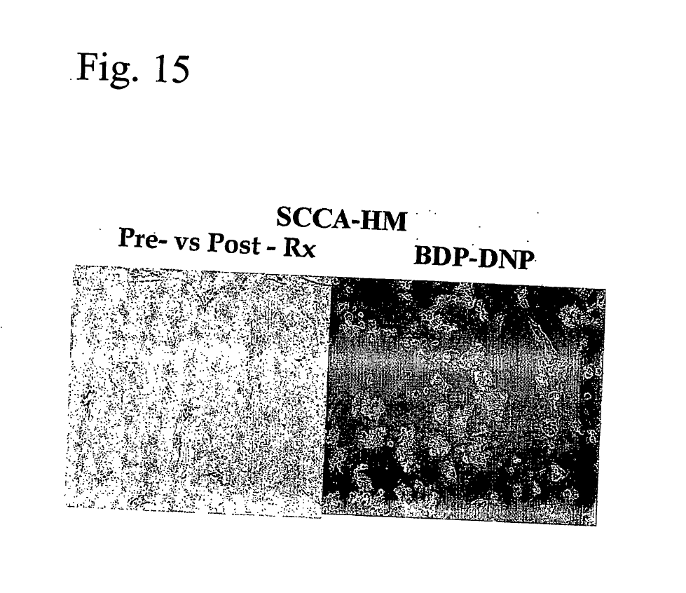

2,6-Dibenzylidenecyclohexanone-2, 4-dinitrophenylhydrazone (BDP-DNP) (Compound 38) is a new hydrazone analog that has 100-fold immune modulating anticancer activities vs. A-007. FIG. 15 describes the impact of BDP-DNP on cell growth for malignant squamous cell cancer (SCCA-HM) growing in culture (pre-post Rx). On the left are large lacey star shaped squamous 15 cancer cells associated with small white clumps of naïve T-lymphocytes. After 24 hours incubation with BDP-DNP (0.4 mcg/mL) in tissue culture media (L. R. Morgan, U.S. Pat. No. 5,270,172) the lymphocytes underwent an impressive stimulation (large white clumps of activated T-lymphocytes) and destruction of the cancer cells. The dark background seen on the right is due to the color of the drug. All the fluffy white cells are large clumps of activated lymphocytes. No cancer cells are detected.

### EXAMPLE 14

### Characteristics of Some Resonance Modulators

As disclosed in the preceding examples, compounds have been found that provide both a diagnostic and therapeutic interface with the immune system, and which independently have anti-tumor and anti-viral activities particularly against HPV and HPV infected cells, such as vaginal and cervical cancers associated with HPV infection.

Compounds have been disclosed that are resonance modulators, that have one or more of the following characteristics, for example at least three or five or all of the following characteristics:

Specific attraction of CD45+ lymphocytes (for example CD45RO+ and CD45RA+ lymphocytes) to the resonance modulator compound, either in culture or when applied to a surface (such as the skin) of a living subject.

Upregulation of CD45RA+ to CD45RO+ lymphocytes, for example when applied to an epithelium of a subject

Possession of inherent electromagnetic properties, for example the ability to form a single line of agglutinated crystals between positively and negatively charged points when the crystals are evaporated, or the emission of electromagnetic energy.

Possession of properties that affect lymphocyte migration, for example inducing aggregation of naïve lymphocytes in culture.

Certain examples of the compounds are also characterized by an ability to increase the amplitude of alternating voltages waves measured from the compound after it is applied to the skin of an individual. In certain examples, the increased amplitude is reduced in subjects having impaired immunity, such as impaired lymphocyte function.

Substantial lack of reactivity.

Substantially no absorption through the skin when applied to the skin, for example it does not satisfy Lipinski's “Rule of 5.” When applied to the skin the agent substantially completely remain on the surface of the skin, without transdermal flux.

Potential resonance modulators can also be selected based on chemical structures that suggest resonance modulation, namely the structural characteristics described earlier in this specification. For example, polyaryl compounds with electronegativity can be selected, such as Casodex®, Naprelan®, Bulexin®, and Bextra®.

Once candidates have been selected as potential resonance modulators, they can be easily assayed to determine whether they induce lymphocyte blastogenesis and activate lymphocytes. Such an assay is found in Morgan et al., Anticancer Research 13:1763-1768 (1993), which is incorporated by reference. See also Janossy et al., Clin. Exp. Immunol. 14:581-596 (1973). Briefly, uptake of [³]thymidine was measured for lymphocytes in culture. Lymphocytes were cultured in RPMI-1640 tissue culture medium supplemented with 10% fetal bovine serum (FBS). Following stimulation with the resonance modulator, tritiated thymidine (2-20 μCi/ml, specific activity 2 Ci/mmol) was added to the cells and incubated with them. The uptake of [³]thymidine was stopped by the addition of cold 10% trichloroacetic acid after a selected period of time, and the radioactivity of the samples measured in a scintillation counter. The % lymphoblast was determined by counting the number of blasts per high powered field (hpf) while % activation was determined by uptake of the tritiated thymidine. The increase voltage was measured with an oscilloscope as in Example 7.

TABLE 4
Prediction of Resonance Modulating Properties

| | | | Increased |
|---|---|---|---|
| Drug/Compound | % Lymphoblast* | % Activation* | voltage** |
| A-007 | 75% | 100% | 54% |
| Compound 38 | 100% | 100% | 69% |
| DNPA (in example 5) | 50% | 75% | 22% |
| Fludamide (Eulexin) | 0% | 0% | 5% |

*The procedure described in Fan, Morgan, et al, (Adoptive Immunotherapy of Advanced Renal Cell Cancer using PHA-stimulated Autologous Lymphocytes, Anticancer Research 16, 230-239, 1993) was used to quantitate the lymphoblasts and lymphocyte activation.
**Measured the increased amplitude with an oscilloscope.

This procedure illustrates a correlation between increased amplitude of voltage signals with enhanced activation of lymphocytes. In particular, greater increases in amplitude were observed with more vigorous enhancement of lymphocyte activation. This assay can be used to quickly screen resonance modulating agent candidates for immune activating activity. For example, using this assay, an increased voltage amplitude of at least 20% (for example an increased amplitude of at least 50%) can be used as a measure of resonance modulation activity on the immune system. Resonance modulation candidates satisfying such criteria (for example an increase in amplitude of at least 20%) are then selected for further use and study.

### EXAMPLE 15

### Topical Preparations

The resonance modulators disclosed herein can be prepared as topical preparations for application to the skin as immunomodulators, anti-viral agents, or anti-neoplastic preparations.

In one example, the resonance modulator comprises 2,6-Dibenzylidenecyclohexanone-2,4-dinitrophenylhydrazone (BDP-DNP). It is placed in a topical preparation for application to an epithelial surface, for example by application to malignant epithelium, such as a urogenital neoplasm, such as an anal, vaginal or cervical neoplasm, such as cervical CIN.

The resonance modulator can be used in methods of treating a tumor (neoplasm) by administering to an affected subject a therapeutically effective amount of the agent to induce regression or elimination of the tumor cells. In particular examples, administering the BDP-DMP comprises applying the BDP-DNP topically to the tumor, although it can also include other forms of administration, such as oral, inhalational, injected or subcutaneous administration.

Having illustrated and described the principles of the invention in several examples, it should be apparent to those skilled in the art that the invention can be modified in specific details without departing from such principles. I claim all modifications coming within the spirit and scope of the following claims.

I claim:

1. A method of stimulating an immune response of a subject in need of immunostimulation, comprising:
administering to the subject in need of immunostimulation a therapeutically effective amount of a resonance modulating compound.

2. The method of claim 1, wherein the subject in need of immunostimulation is a subject who has laboratory evidence of impaired immunity.

3. The method of claim 1, wherein the resonance modulating compound is a phenylhydrazone.

4. The method of claim 3, wherein the resonance modulating compound is a nitropheylhydrazone.

5. The method of claim 4, wherein the resonance modulating compound is a di-nitrophenylhydrazone.

6. The method of claim 5, wherein the resonance modulating compound is a 2,4-di-nitrophenylhydrazone.

7. The method of claim 3, wherein the resonance modulating compound comprises: 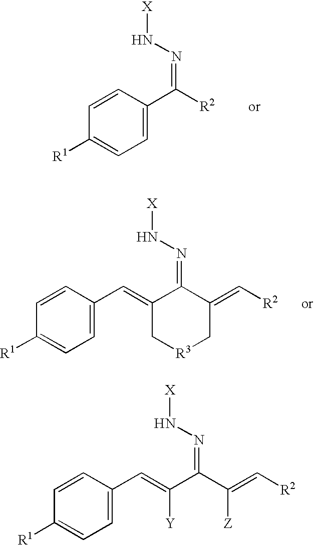
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄, C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³═—O—, —S—, —CH₂—, —N—,—, —CHA- and —CHOA-; where A=aryl, ester, amide, lipid, carbohydrate, or peptide;
Y═H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

8. The method of claim 7, wherein the resonance modulating compound is

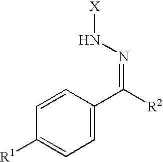

9. The method of claim 7, wherein the resonance modulating compound is

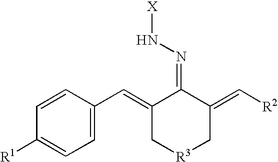

10. The method of claim 7, wherein the resonance modulating compound is

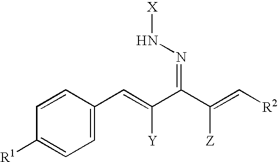

11. The method of claim 8, wherein R¹ is OH, R² is C₆H₄OH and X is C₆H₃-2,4(NO₂)₂.

12. The method of claim 9, wherein the compound is 2,6-dibenzylidenecyclohexanone-2,4-dinitrophenylhydrazone.

13. The method of claim 1, wherein administering the compound comprises applying the compound to an epithelial surface of the subject.

14. The method of claim 13, wherein applying the compound to the epithelial surface of the subject comprises applying the compound to the surface of skin.

15. The method of claim 13, wherein applying the compound to an epithelial surface of the subject comprises introducing the compound into the skin.

16. The method of claim 1, wherein the method of stimulating an immune response comprises a method of treating a tumor by stimulating the immune response of the subject.

17. The method of claim 14, wherein applying the compound to the skin of the subject comprises applying the compound to an area of skin that is over or adjacent a tumor to stimulate the immune response of the subject.

18. The method of claim 17, wherein the tumor is a malignant tumor.

19. The method of claim 18, wherein the tumor is a metastasis.

20. The method of claim 1, further comprising enhancing immune stimulation by exposing the compound to an electromagnetic field that induces increased resonance modulation of the compound.

21. The method of claim 20, wherein the electromagnetic field is produced by a magnetic probe.

22. The method of claim 20, wherein the electromagnetic field is produced by a current flowing between two electrodes.

23. The method of claim 1, further comprising monitoring an amplitude of a waveform generated by resonance modulation of the compound to determine a response of the subject's immune system to the compound.

24. The method of claim 23, further comprising exposing the compound to an electromagnetic field to induce increased resonance modulation of the compound if the amplitude is below a therapeutically desired amplitude.

25. The method of claim 1, wherein the method of stimulating an immune response comprises a method of stimulating an immune response by or against a cell that expresses an extracellular membrane-bound protein tyrosine phosphatase.

26. The method of claim 25, wherein the cell is a T lymphocyte, and the method of stimulating the immune response comprises stimulating the immune response by the T-lymphocyte against an antigen.

27. The method of claim 25, wherein the cell is a cell that is infected with a CD45+ virus, and the method of stimulating the immune response comprises stimulating the immune response against the cell that is infected with the virus.

28. The method of claim 27, wherein the virus is a papillomavirus or a retrovirus.

29. The method of claim 28, wherein the virus is a papillomavirus.

30. The method of claim 29, wherein the virus is a human papillomavirus.

31. The method of claim 28, wherein the virus is a retrovirus.

32. The method of claim 31, wherein the virus is a human immunodeficiency virus.

33. The method of claim 25, wherein the cell is a cell that is infected with a virus comprising an active extracellular membrane-bound protein tyrosine phosphatase, and the method of stimulating the immune response comprises stimulating the immune response against the cell that is infected with the virus.

34. A method of enhancing an immune response in a subject, comprising applying to the skin of the subject a therapeutically effective amount of a compound 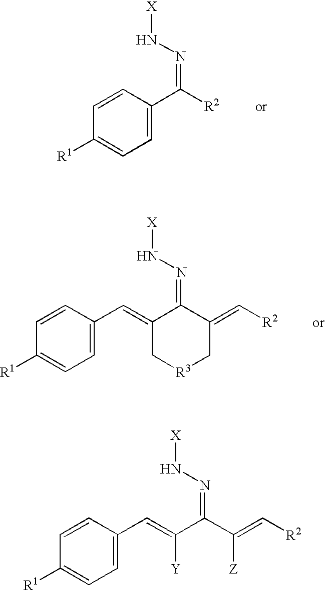
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³═—O—, —S—, —CH₂—, —N—,—, —CHA- and —CHOA-; where A=aryl, ester, amide, lipid, carbohydrate, or peptide;
Y═H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

35. A method of monitoring an immune response of a subject, comprising:
placing a resonance modulating compound in contact with the subject to produce inherent electromagnetic waveforms characteristic of the resonance modulating compound that indicate an immune state of the subject; and
monitoring waveforms produced by the compound for waveforms associated with altered immune function.

36. The method of claim 35, wherein monitoring the waveforms comprises detecting voltage changes across the compound over time, without introducing an external current or voltage potential.

37. The method of claim 35, wherein monitoring the waveforms associated with altered immune function comprise detecting waveforms having a decreased amplitude.

38. The method of claim 35, wherein monitoring waveforms comprises:
positioning electrodes to detect a voltage potential across the compound when the compound is in contact with the subject;
detecting electrical potential waveforms associated with altered immune function; and
providing a diagnostic or therapeutic intervention in response to detection of the waveforms associated with altered immune function.

39. The method of claim 38, wherein the intervention comprises a diagnostic intervention.

40. The method of claim 39, wherein the diagnostic intervention comprises an intervention designed to detect an infectious or neoplastic condition.

41. The method of claim 38, wherein the intervention comprises a therapeutic intervention.

42. The method of claim 41, wherein the therapeutic intervention comprises administration of an anti-infectious or anti-neoplastic therapy.

43. The method of claim 42, wherein the therapeutic intervention comprises modulating resonance activity of the compound.

44. The method of claim 43, wherein modulating resonance activity of the compound comprises applying an induced electromagnetic field to the, compound that increases resonance modulation of the compound.

45. The method of claim 35, wherein the resonance modulating compound is a phenylhydrazone.

46. The method of claim 45, wherein the resonance modulating compound is a polyaryl phenylhydrazone.

47. The method of claim 35, wherein the resonance modulating compound is 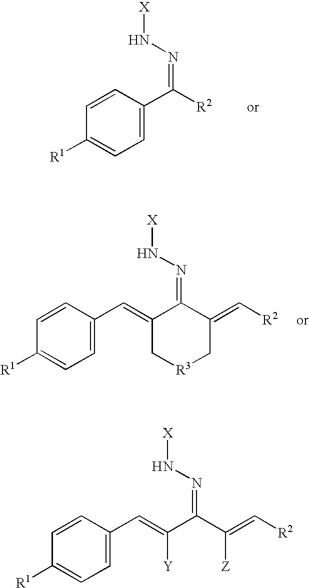
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂ C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³═—O—, —S—, —CH₂—, —N—, —, —CHA- and —CHOA-; where A=aryl, ester, amide, lipid, carbohydrate, or peptide;
Y═H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

48. A method of treating a tumor, comprising:
exposing the tumor to a therapeutically effective dose of a resonance modulating compound; and
applying an external electromagnetic field to the compound to increase a resonance modulation of the compound and thereby increase an immunostimulant effect of the compound to treat the tumor.

49. The method of claim 48, wherein the resonance modulating compound is a phenylhydrazone.

50. The method of claim 49, wherein the resonance modulating compound is 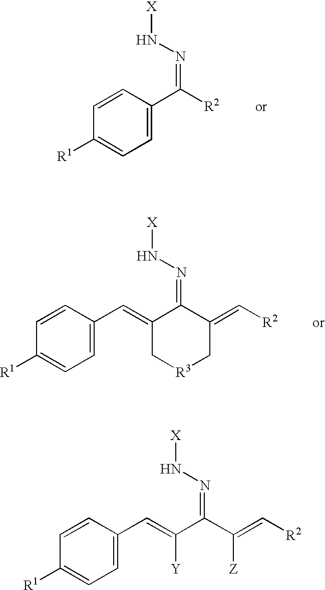
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³═—O—, —S—, —CH₂—, —N—,—, —CHA- and —CHOA-; where A=aryl, ester, amide, lipid, carbohydrate, or peptide;
Y═H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

51. A method of concentrating lymphocytes from biological tissue, comprising exposing the biological tissue to an effective amount of a resonance modulating compound.

52. The method of claim 51, wherein the resonance modulating compound is 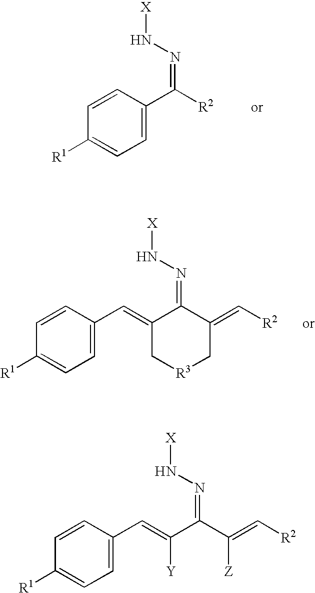
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³═—O—, —S—, —CH₂—, —N—, —, —CHA- and —CHOA-; where A=aryl, ester, amide, lipid, carbohydrate, or peptide;
Y═H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

53. The method of claim 52, wherein exposing the living organism to the effective amount of the compound comprises applying the compound to skin of the living organism.

54. The method of claim 53, wherein applying the compound to the skin comprises applying a topical preparation of the compound to the surface of the skin.

55. A method of treating an infection in a subject, comprising:
administering to the subject a therapeutically effective amount of a resonance modulating compound that interacts with a PTP extracellular receptor of a cell to activate the receptor.

56. The method of claim 55, further comprising applying an external electromagnetic field to the compound to increase a resonance modulation of the compound, and thereby increase an immunostimulant effect of the compound to treat the infection.

57. The method of claim 55, wherein the infection is a virus infection.

58. The method of claim 57, wherein the virus is a papillomavirus or a retrovirus.

59. The method of claim 58, wherein the virus is a papillomavirus.

60. The method of claim 59, wherein the virus is a human papillomavirus.

61. The method of claim 57, wherein the virus is a retrovirus.

62. The method of claim 61, wherein the virus is a human immunodeficiency virus.

63. The method of claim 55, wherein the infection is a bacterial infection.

64. The method of claim 55, wherein the cell is a T-lymphocyte.

65. The method of claim 64, wherein the cell is a CD45+ lymphocyte.

66. The method of claim 55, wherein the cell is a CD45+ pathogen.

67. The method of claim 66, wherein the pathogen is human papillomavirus.

68. The method of claim 55, further comprising enhancing interaction between the resonance modulating compound and the receptor by exposing the compound and cell to ultraviolet radiation.

69. The method of claim 55, wherein administering the compound to the subject comprises applying the compound topically to the subject.

70. The method of claim 69, wherein applying the compound to the subject comprises applying the compound to dysplastic or metaplastic epithelium.

71. The method of claim 70, wherein the epithelium is urogenital epithelium.

72. The method of claim 71, wherein the urogenital epithelim is vaginal, cervical or anal epithelium.

73. The method of claim 55, wherein the compound comprises 2,6-dibenzylidenecyclohexanone-2,4-dinitrophenylhydrazone.

74. The method of claim 69, wherein applying the compound to the subject comprises applying the compound to a squamous cell cancer.

75. The method of claim 74, wherein the tumor is an anal squamous cell cancer.

76. The method of claim 74, wherein the tumor is a squamous cell vaginal or cervical cancer.

77. The method of claim 69, wherein administering the compound comprises applying the compound to skin of the subject.

78. The method of claim 69, wherein applying the compound comprises applying the compound intra-anally.

79. The method of claim 69, wherein applying the compound comprises applying the compound intra-vaginally.

80. The method of claim 55, wherein the compound comprises a topical gel.

81. The method of claim 80, wherein the topical gel comprises 0.25% of the compound.

82. The method of claim 78, wherein applying the compound topically to the subject comprises applying the compound at least daily for a sufficient period to produce a therapeutic effect.

83. The method of claim 85, wherein applying the compound comprises applying the compound at least daily for at least five days.

84. The method of claim 55, wherein the therapeutically effective amount of the compound is at least 2 grams of a topical gel per day, wherein the gel comprises at least about 0.25% of the compound.

85. A compound, comprising 2,6-Dibenzylidenecyclohexanone-2,4-dinitrophenylhydrazone (BDP-DNP).

86. A method of treating a neoplasm, comprising administering an anti-neoplastic therapeutic amount of BDP-DNP to a subject having a neoplasm.

87. The method of claim 86, wherein the neoplasm is a urogenital neoplasm.

88. The method of claim 87, wherein the neoplasm is an anal, vaginal or cervical neoplasm.

89. The method of claim 86, wherein administering the BDP-DMP comprises applying the BDP-DNP to the tumor.

90. The method of claim 89, wherein applying the BDP-DNP comprises applying the BDP-DNP topically to or over the tumor.

91. The compound of claim 86, comprising a topical preparation of BDP-DNP.

92. A method of activating a cellular protein tyrosine phosphatase present in a cell membrane, comprising exposing the cell to an amount of a resonance modulator compound sufficient to activate the protein tyrosine phosphatase.

93. The method of claim 92, wherein exposing the cell to the amount of the resonance modulator comprises applying the resonance modulator topically to or adjacent the cell.

94. The method of claim 92, wherein the protein tyrosine phosphatase is a CD45 receptor on a lymphocyte.
